(19) United States
(12) Patent Application Publication
(10) Pub. No.: US 2025/0031964 A1
(43) Pub. Date: Jan. 30, 2025

(54) SYSTEMS, DEVICES, AND METHODS OF USING BLOCKCHAIN FOR TRACKING PATIENT IDENTIFICATION

(71) Applicant: ABBOTT DIABETES CARE INC., Alameda, CA (US)

(72) Inventors: Luca BIROLINI, Southlake, TX (US); John M. SCHULLIAN, Cary, IL (US)

(73) Assignee: ABBOTT DIABETES CARE INC., Alameda, CA (US)

(21) Appl. No.: 18/709,732

(22) PCT Filed: Nov. 4, 2022
(86) PCT No.: PCT/US2022/048915

§ 371 Date: May 13, 2024

Related U.S. Application Data
(60) Provisional application No. 63/279,015, filed on Nov. 12, 2021.

(51) Int. Cl. A61B 5/00; A61B 5/145; G16H 10/60

(52) U.S. Cl. CPC A61B 5/0022; A61B 5/14532; A61B 5/14546; A61B 5/742; G16H 10/60

(57) ABSTRACT

A system for bi-directional communication of patient data can include a first database having a first record including first data associated with a personal identification of a patient, a second database having a second record including second data associated with a user identification of the patient; and one or more processors configured to: pair the first data and the second data based upon a shared data item contained in the first record and the second record, and display a combination of the first data paired with the second data. A blockchain is used to paid the first and second records associated with different user identifications of the same patient.

### CROSS-REFERENCE TO RELATED APPLICATIONS

This application claims the benefit, under 35 U.S.C. § 119 (e), of U.S. Provisional Patent Application No. 63/279,015, filed Nov. 12, 2021, which is incorporated herein by reference in its entirety and for all purposes.

### FIELD

The subject matter described herein relates generally to systems and methods of bi-directional communication of patient data.

### BACKGROUND

The detection and/or monitoring of analyte levels, such as glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc., or the like, can be important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Additionally, certain patient information, particularly as it relates to laboratory test results, currently resides at various healthcare care organization's (HCO) local computer networks (e.g., electronic medical/health records). Such information is recorded and stored on EMR systems using patient identification that is unique to each HCO. Similarly, analyte monitoring systems often store analyte measurements on a centralized database using user identification (e.g., username, email address, etc.).

Thus, needs exist for systems and methods for bi-directional communication so that patient data from HCOs can be paired with data from analyte measurement systems.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

The achieve these and other advantages and in accordance with the purpose of the disclosed subject matter, as embodied and broadly described, the disclosed subject matter is directed to systems and methods for bi-direction communication of patient data. According to an embodiment, a system for bi-directional communication can include a first database having a first record including first data associated with a personal identification of a patient, a second database having a second record including second data associated with a user identification of the patient, and one or more processors configured to: pair the first data and the second data based upon a shared data item contained in the first record and the second record, and display a combination of the first data paired with the second data.

As embodied herein, the first database can be an electronic medical record system. The first data can be laboratory measured HbA1c. As embodied herein, the second database can include an analyte monitoring system data service. As embodied herein, the second data can include glucose levels measured by, for example, an analyte monitoring system. As embodied herein, the shared data item can include an email address.

As embodied herein, the one or more processors can be configured to receive a request to read, write, edit, or delete a resource data in the first or second database, wherein the request can be formatted according to a Fast Healthcare Interoperability Resources (FHIR) standard and FHIR extensions embodying a healthcare provider directory (HPD) standard, or H7. As embodied herein, the one or more processors can be further configured to generate a notification based upon the first data paired with the second data. Further, the notification can be displayed as the combination of the first data paired with the second data.

As embodied herein, the one or more processors can be further configured to perform a calculation based upon the first data paired with the second data. Further, the calculation can include calculation of a glucose derived A1c. Alternatively, the calculation can also include calculation of a personalized HbA1c.

In accordance with the disclosed subject matter, some embodiments disclose a method of bi-directional communication of patient data. The method can include the steps of receiving a first data associated with a personal identification, using one or more processors, from a first database, receiving a second data associated with a user identification, using the one or more processors, from a second database, pairing, using the one or more processors, the first data and the second data based upon a shared data item contained in the first record and the second record, and displaying, using one or more processors, a combination of the first data and the second data.

As embodied herein, the first database can be an electronic medical record system. As embodied herein, the first data can be laboratory measured HbA1c. Alternatively, or in addition, as embodied herein, the second database can include an analyte monitoring system data service. As embodied herein, the second data can include glucose levels measured by an analyte monitoring system. As embodied herein, the shared data item can include an email address. A blockchain further allows for linking of different patient IDs and user IDs and can be used alongside the databases.

As embodied herein, the method can further comprise, generating, using the one or more processors, a notification based upon the first data paired with the second data. As embodied herein, the method can further comprise performing, using the one or more processors, a calculation based upon the first data paired with the second data. Further, the calculation can include calculation of a glucose derived. Alternatively, the calculation can further include calculation of a personalized HbA1c.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 2D to 2I are example embodiments of GUIs comprising sensor results interfaces.

FIGS. 3A to 3F are example embodiments of GUIs comprising time-in-ranges interfaces.

FIGS. 4A to 4O are example embodiments of GUIs comprising analyte level and trend alert interfaces.

FIGS. 5A and 5B are example embodiments of GUIs comprising sensor usage interfaces.

FIGS. 5C to 5F are example embodiments of report GUIs including sensor usage information.

FIGS. 5G-5L are example embodiments of GUIs relating to an analyte monitoring software application.

FIGS. 6A and 6B are flow diagrams depicting example embodiments of methods for data backfilling in an analyte monitoring system.

FIG. 6C is a flow diagram depicting an example embodiment of a method for aggregating disconnect and reconnect events in an analyte monitoring system.

FIG. 7 is a flow diagram depicting an example embodiment of a method for failed or expired sensor transmissions in an analyte monitoring system.

FIGS. 8A and 8B are flow diagrams depicting example embodiments of methods for data merging in an analyte monitoring system.

FIGS. 8C to 8E are graphs depicting data at various stages of processing according to an example embodiment of a method for data merging in an analyte monitoring system.

FIG. 9A is a flow diagram depicting an example embodiment of a method for sensor transitioning in an analyte monitoring system.

FIGS. 9B to 9D are example embodiments of GUIs to be displayed according to an example embodiment of a method for sensor transitioning in an analyte monitoring system.

FIG. 10A is a flow diagram depicting an example embodiment of a method for generating a sensor insertion failure system alarm.

FIGS. 10B to 10D are example embodiments of GUIs to be displayed according to an example embodiment of a method for generating a sensor insertion failure system alarm.

FIG. 11A is a flow diagram depicting an example embodiment of a method for generating a sensor termination system alarm.

FIGS. 11B to 11D are example embodiments of GUIs to be displayed according to an example embodiment of a method for generating a sensor termination system alarm.

FIGS. 12A-12C are example embodiments of systems for bi-directional communication of patient data.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of this disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of this application. Nothing herein is to be construed as an admission that this disclosure is not entitled to antedate such publication by virtue of prior disclosure. Further, the dates of publication provided may be different from the actual publication dates which may need to be independently confirmed.

Generally, embodiments of this disclosure include GUIs and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely noninvasive.

Furthermore, for each and every embodiment of a method disclosed herein, systems and devices capable of performing each of those embodiments are covered within the scope of this disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs for analyte monitoring systems, wherein the GUIs are highly intuitive, user-friendly, and provide for rapid access to physiological information of a user. According to some embodiments, a Time-in-Ranges GUI of an analyte monitoring system is provided, wherein the Time-in-Ranges GUI comprises a plurality of bars or bar portions, wherein each bar or bar portion indicates an amount of time that a user's analyte level is within a predefined analyte range correlating with the bar or bar portion. According to another embodiment, an Analyte Level/Trend Alert GUI of an analyte monitoring system is provided, wherein the Analyte Level/Trend Alert GUI comprises a visual notification (e.g., alert, alarm, pop-up window, banner notification, etc.), and wherein the visual notification includes an alarm condition, an analyte level measurement associated with the alarm condition, and a trend indicator associated with the alarm condition. In sum, these embodiments provide for a robust, user-friendly interfaces that can increase user engagement with the analyte monitoring system and provide for timely and actionable responses by the user, to name a few advantages.

In addition, a number of embodiments described herein provide for improved digital interfaces for analyte monitoring systems. According to some embodiments, improved methods, as well as systems and device relating thereto, are provided for data backfilling, aggregation of disconnection and reconnection events for wireless communication links, expired or failed sensor transmissions, merging data from multiple devices, transitioning of previously activated sensors to new reader devices, generating sensor insertion failure system alarms, and generating sensor termination system alarms. Collectively and individually, these digital interfaces improve upon the accuracy and integrity of analyte data being collected by the analyte monitoring system, the flexibility of the analyte monitoring system by allowing users to transition between different reader devices, and the alarming capabilities of the analyte monitoring system by providing for more robust inter-device communications during certain adverse conditions, to name only a few. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

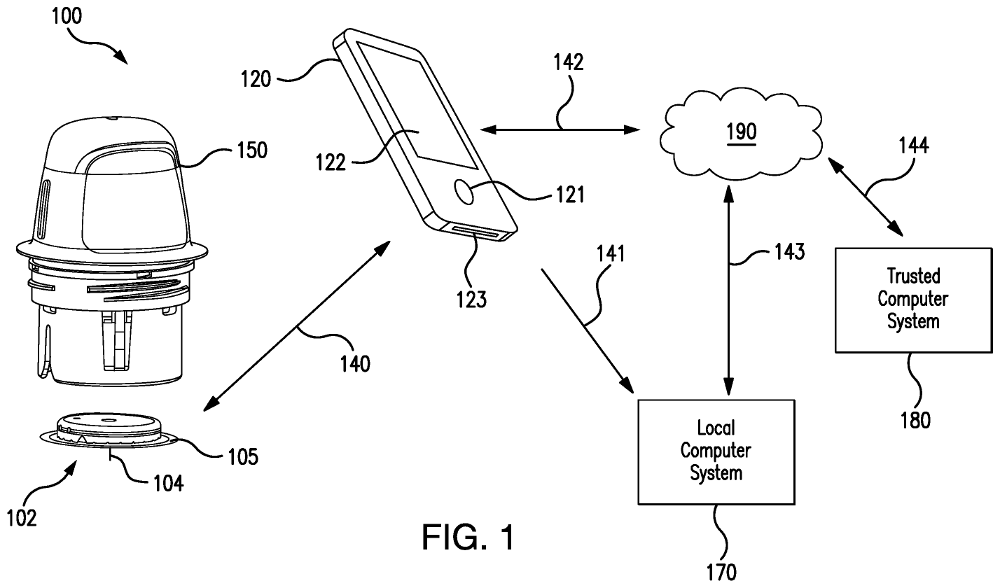

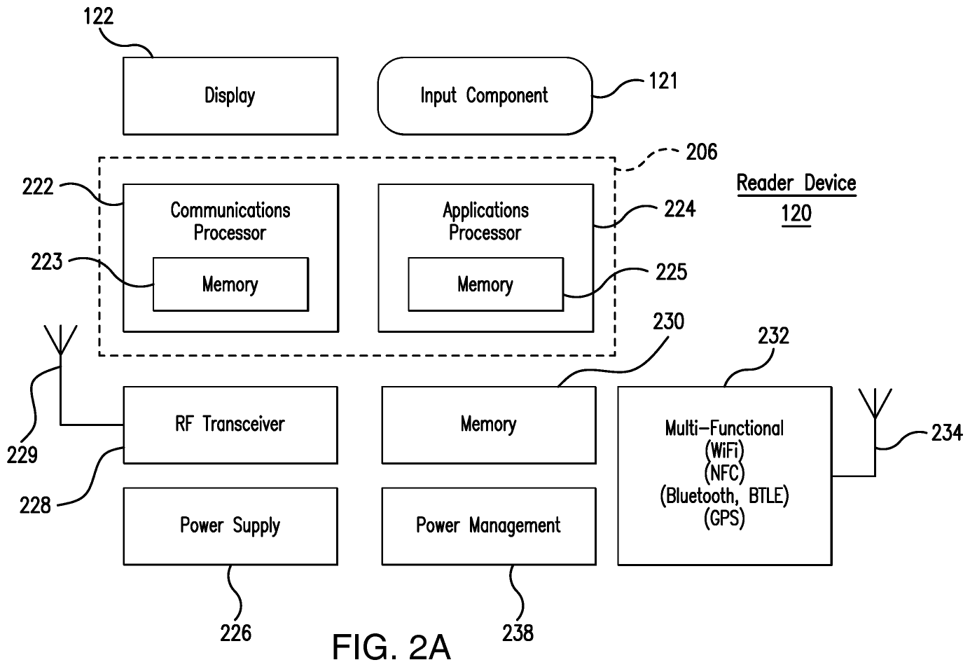

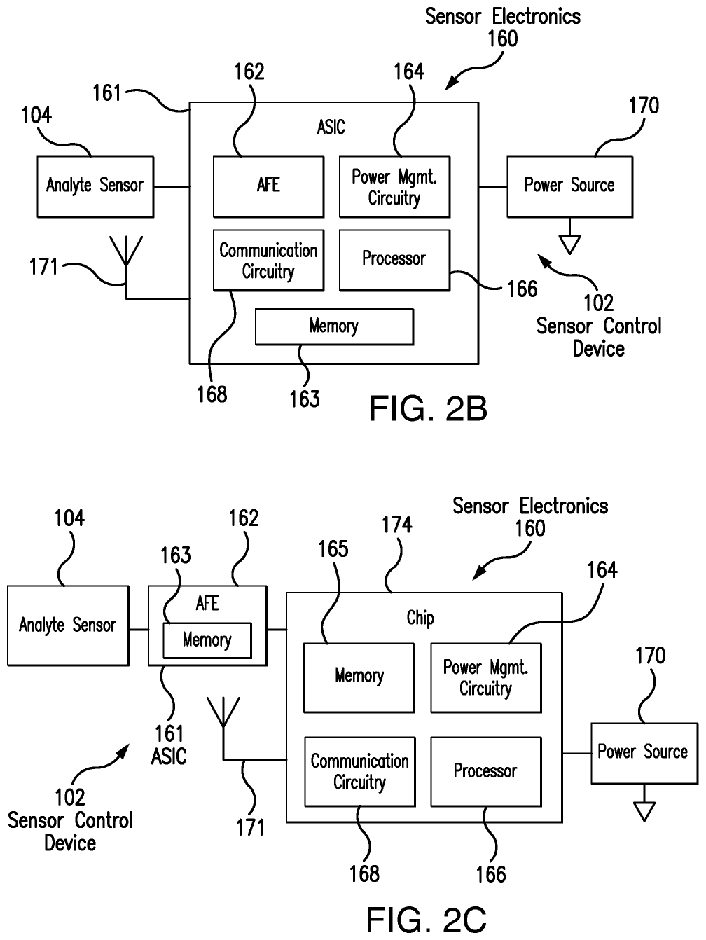

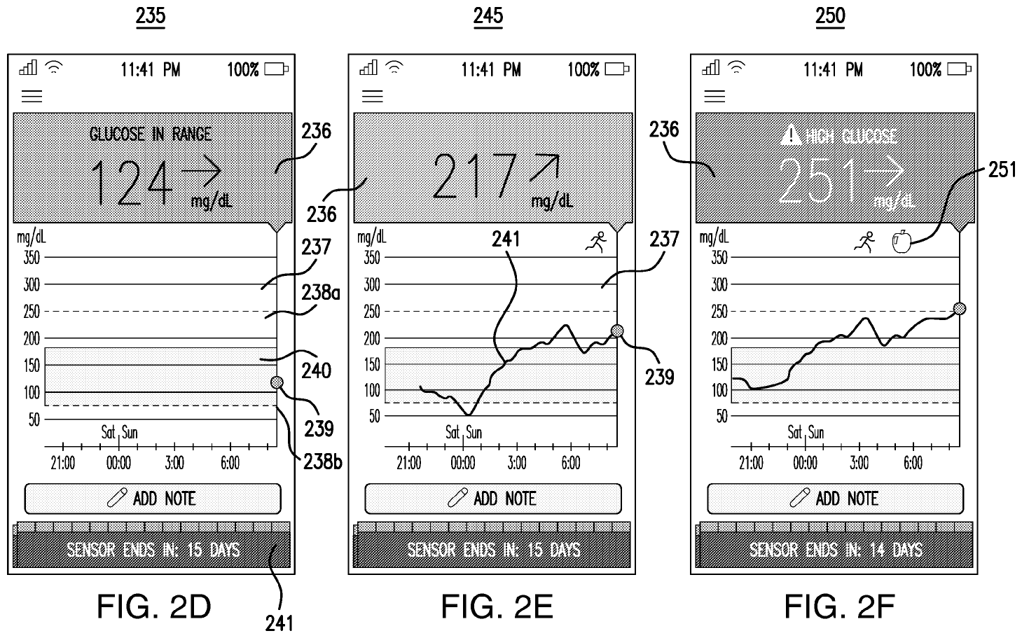

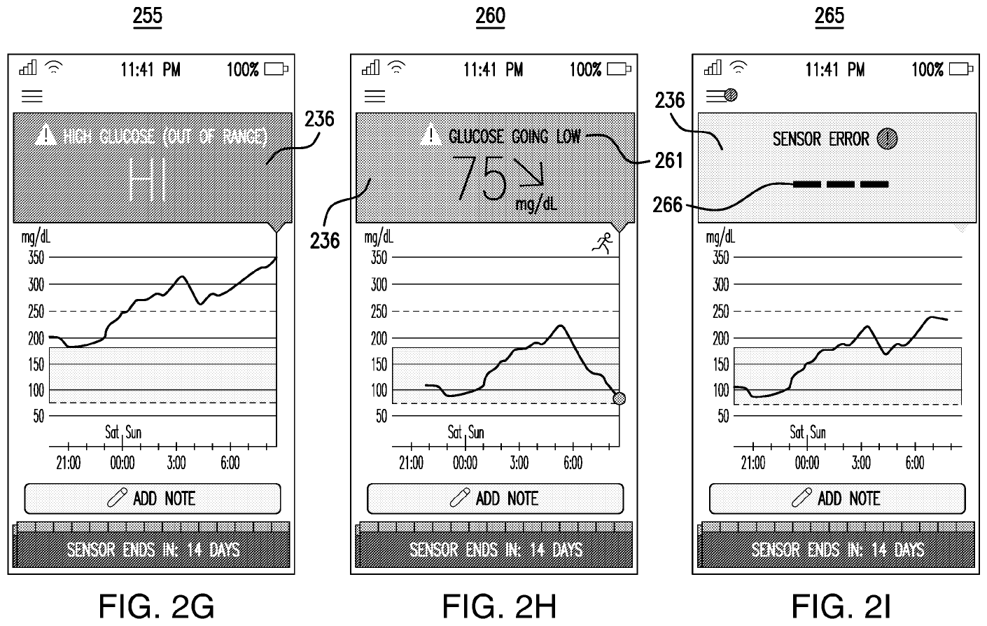

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage (e.g., storage of analyte measurement data received from reader device), report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

Additional details of suitable analyte monitoring devices, systems, methods, components and the operation thereof along with related features are set forth in U.S. Pat. No. 9,913,600 to Taub et. al., International Publication No. WO2018/136898 to Rao et. al., International Publication No. WO2019/236850 to Thomas et. al., and U.S. Patent Publication No. 2020/01969191 to Rao et al., each of which is incorporated by reference in its entirety herein.

### Example Embodiment of Reader Device

FIG. 2A is a block diagram depicting an example embodiment of a reader device 120, which, in some embodiments, can comprise a smart phone or a smart watch. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with one or more antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed-in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems. As an initial matter, it will be understood by those of skill in the art that the GUIs described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

### Example Embodiments of Sensor Results Interfaces

FIGS. 2D to 2I depict example embodiments of sensor results interfaces or GUIs for analyte monitoring systems. In accordance with the disclosed subject matter, the sensor results GUIs described herein are configured to display analyte data and other health information through a user interface application (e.g., software) installed on a reader device, such as a smart phone or a receiver, like those described with respect to FIG. 2B. Those of skill in the art will also appreciate that a user interface application with a sensor results interface or GUI can also be implemented on a local computer system or other computing device (e.g., wearable computing devices, smart watches, tablet computer, etc.).

Referring first to FIG. 2D, sensor results GUI 235 depicts an interface comprising a first portion 236 that can include a numeric representation of a current analyte concentration value (e.g., a current glucose value), a directional arrow to indicate an analyte trend direction, and a text description to provide contextual information such as, for example, whether the user's analyte level is in range (e.g., “Glucose in Range”). First portion 236 can also comprise a color or shade that is indicative of an analyte concentration or trend. For example, as shown in FIG. 2D, first portion 236 is a green shade, indicating that the user's analyte level is within a target range. According to some embodiments, for example, a red shade can indicate an analyte level below a low analyte level threshold, an orange shade can indicate an analyte level above a high analyte level threshold, and an yellow shade can indicate an analyte level outside a target range. In addition, according to some embodiments, sensor results GUI 235 also includes a second portion 237 comprising a graphical representation of analyte data. In particular, second portion 237 includes an analyte trend graph reflecting an analyte concentration, as shown by the y-axis, over a predetermined time period, as shown by the x-axis. In some embodiments, the predetermined time period can be shown in five-minute increments, with a total of twelve hours of data. Those of skill in the art will appreciate, however, that other time increments and durations of analyte data can be utilized and are fully within the scope of this disclosure. Second portion 237 can also include a point 239 on the analyte trend graph to indicate the current analyte concentration value, a shaded green area 240 to indicate a target analyte range, and two dotted lines 238a and 238b to indicate, respectively, a high analyte threshold and a low analyte threshold. According to some embodiments, GUI 235 can also include a third portion 241 comprising a graphical indicator and textual information representative of a remaining amount of sensor life.

Referring next to FIG. 2E, another example embodiment of a sensor results GUI 245 is depicted. In accordance with the disclosed subject matter, first portion 236 is shown in a yellow shade to indicate that the user's current analyte concentration is not within a target range. In addition, second portion 237 includes: an analyte trend line 241 which can reflect historical analyte levels over time and a current analyte data point 239 to indicate the current analyte concentration value (shown in yellow to indicate that the current value is outside the target range).

According to another aspect of the embodiments, data on sensor results GUI 245 is automatically updated or refreshed according to an update interval (e.g., every second, every minute, every 5 minutes, etc.). For example, according to many of the embodiments, as analyte data is received by the reader device, sensor results GUI 245 will update: (1) the current analyte concentration value shown in first portion 236, and (2) the analyte trend line 241 and current analyte data point 239 show in second portion 237. Furthermore, in some embodiments, the automatically updating analyte data can cause older historical analyte data (e.g., in the left portion of analyte trend line 241) to no longer be displayed.

FIG. 2F is another example embodiment of a sensor results GUI 250. According to the depicted embodiment, sensor results GUI 250 includes first portion 236 which is shown in an orange shade to indicate that the user's analyte levels are above a high glucose threshold (e.g., greater than 250 mg/dL). Sensor results GUI 250 also depicts health information icons 251, such as an exercise icon or an apple icon, to reflect user logged entries indicating the times when the user had exercised or eaten a meal.

FIG. 2G is another example embodiment of a sensor results GUI 255. According to the depicted embodiments, sensor results GUI 255 includes first portion 236 which is also shown in an orange shade to indicate that the user's analyte levels are above a high glucose threshold. As can be seen in FIG. 2G, first portion 236 does not report a numeric value but instead displays the text “HI” to indicate that the current analyte concentration value is outside a glucose reporting range high limit. Although not depicted in FIG. 2G, those of skill in the art will understand that, conversely, an analyte concentration below a glucose reporting range low limit will cause first portion 236 not to display a numeric value, but instead, the text “LO”.

FIG. 2H is another example embodiment of a sensor results GUI 260. According to the depicted embodiments, sensor results GUI 260 includes first portion 236 which is shown in a green shade to indicate that the user's current analyte level is within the target range. In addition, according to the depicted embodiments, first portion 236 of GUI 260 includes the text, “GLUCOSE GOING LOW,” which can indicate to the user that his or her analyte concentration value is predicted to drop below a predicted low analyte level threshold within a predetermined amount of time (e.g., predicted glucose will fall below 75 mg/dL within 15 minutes). Those of skill in the art will understand that if a user's analyte level is predicted to rise above a predicted high analyte level threshold within a predetermined amount of time, sensor results GUI 260 can display a “GLUCOSE GOING HIGH” message.

FIG. 2I is another example embodiment of a sensor results GUI 265. According to the depicted embodiments, sensor results GUI 265 depicts first portion 236 when there is a sensor error. In accordance with the disclosed subject matter, first portion 236 includes three dashed lines 266 in place of the current analyte concentration value to indicate that a current analyte value is not available. In some embodiments, three dashed lines 266 can indicate one or more error conditions such as, for example, (1) a no signal condition; (2) a signal loss condition; (3) sensor too hot/cold condition; or (4) a glucose level unavailable condition. Furthermore, as can be seen in FIG. 2I, first portion 236 comprises a gray shading (instead of green, yellow, orange, or red) to indicate that no current analyte data is available. In addition, according to another aspect of the embodiments, second portion 237 can be configured to display the historical analyte data in the analyte trend graph, even though there is an error condition preventing the display of a numeric value for a current analyte concentration in first portion 236. However, as shown in FIG. 2I, no current analyte concentration value data point is shown on the analyte trend graph of second portion 237.

### Example Embodiments of Time-in-Ranges Interfaces

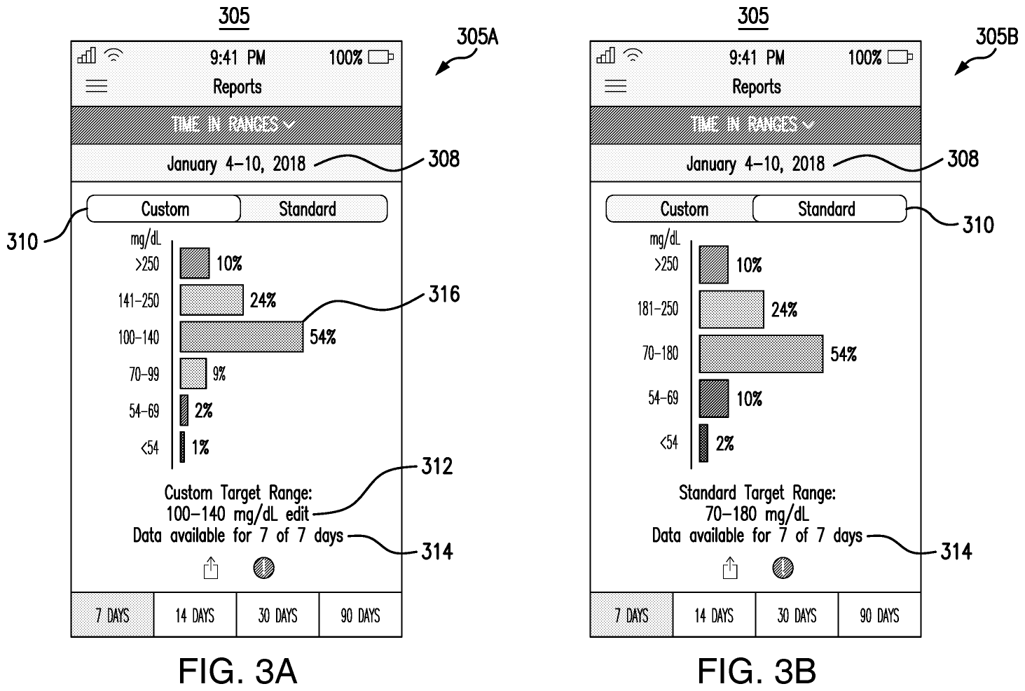

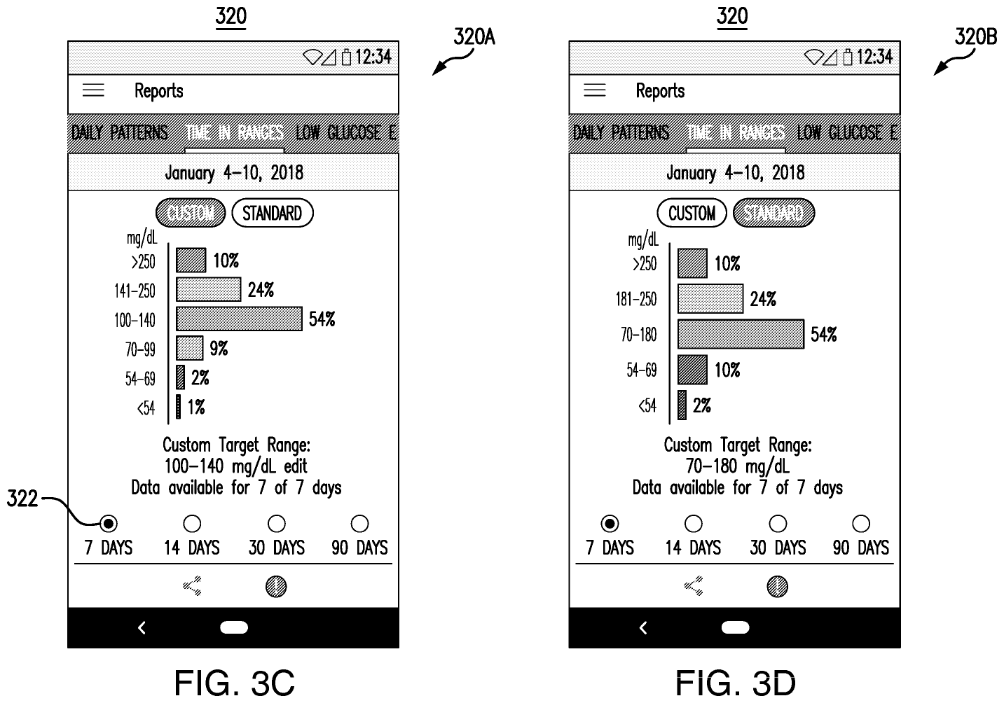

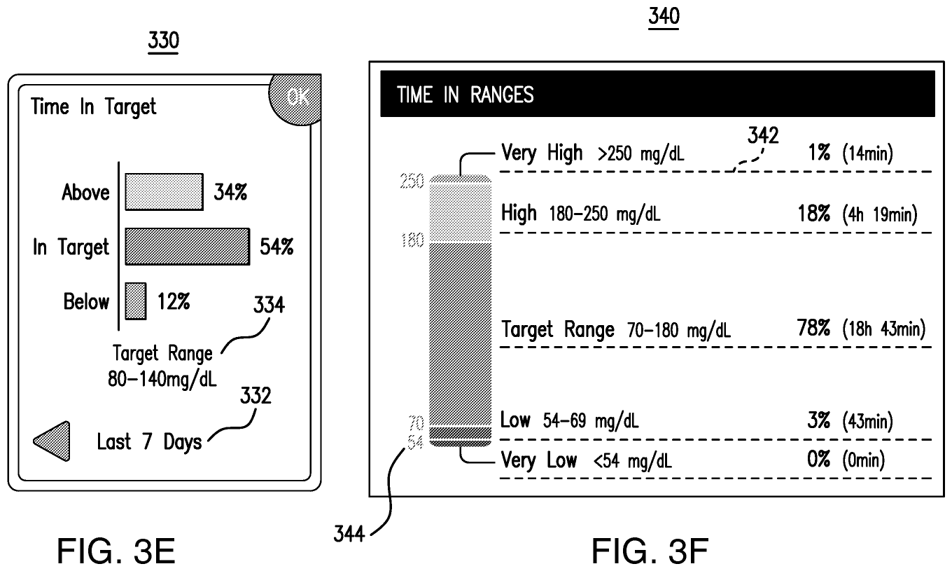

FIGS. 3A to 3F depict example embodiments of GUIs for analyte monitoring systems. In particular, FIGS. 3A to 3F depict Time-in-Ranges (also referred to as Time-in-Range and/or Time-in-Target) GUIs, each of which comprise a plurality of bars or bar portions, wherein each bar or bar portion indicates an amount of time that a user's analyte level is within a predefined analyte range correlating with the bar or bar portion. In some embodiments, for example, the amount of time can be expressed as a percentage of a predefined amount of time.

Turning to FIGS. 3A and 3B, an example embodiment of a Time-in-Ranges GUI 305 is shown, wherein Time-in-Ranges GUI 305 comprises a “Custom” Time-in-Ranges view 305A and a “Standard” Time-in-Ranges view 305B, with a slidable element 310 that allows the user to select between the two views. In accordance with the disclosed subject matter, Time-in-Ranges views 305A, 305B can each comprise multiple bars, wherein each bar indicates an amount of time that a user's analyte level is within a predefined analyte range correlating with the bar. In some embodiments, Time-in-Ranges views 305A, 305B further comprise a date range indicator 308, showing relevant dates associated with the displayed plurality of bars, and a data availability indicator 314, showing the period(s) of time in which analyte data is available for the displayed analyte data (e.g., “Data available for 7 of 7 days”).

Referring to FIG. 3A, “Custom” Time-in-Ranges view 305A includes six bars comprising (from top to bottom): a first bar indicating that the user's glucose range is above 250 mg/dL for 10% of a predefined amount of time, a second bar indicating that the user's glucose range is between 141 and 250 mg/dL for 24% of the predefined amount of time, a third bar 316 indicating that the user's glucose range is between 100 and 140 mg/dL for 54% of the predefined amount of time, a fourth bar indicating that the user's glucose range is between 70 and 99 mg/dL for 9% of the predefined amount of time, a fifth bar indicating that the user's glucose range is between 54 and 69 mg/dL for 2% of the predefined amount of time, and a sixth bar indicating that the user's glucose range is less than 54 mg/dL for 1% of the predefined amount of time.

Those of skill in the art will recognize that the glucose ranges and percentages of time associated with each bar can vary depending on the ranges defined by the user and the available analyte data of the user. Furthermore, although FIGS. 3A and 3B show a predefined amount of time 314 equal to seven days, those of skill in the art will appreciate that other predefined amounts of time can be utilized (e.g., one day, three days, fourteen days, thirty days, ninety days, etc.), and are fully within the scope of this disclosure.

According to another aspect of the embodiments, “Custom” Time-in-Ranges view 305A also includes a user-definable custom target range 312 that includes an actionable “edit” link that allows a user to define and/or change the custom target range. As shown in “Custom” Time-in-Ranges view 305A, the custom target range 312 has been defined as a glucose range between 100 and 140 mg/dL and corresponds with third bar 316 of the plurality of bars. Those of skill in the art will also appreciate that, in other embodiments, more than one range can be adjustable by the user, and such embodiments are fully within the scope of this disclosure.

Referring to FIG. 3B, “Standard” Time-in-Ranges view 305B includes five bars comprising (from top to bottom): a first bar indicating that the user's glucose range is above 250 mg/dL for 10% of a predefined amount of time, a second bar indicating that the user's glucose range is between 181 and 250 mg/dL for 24% of the predefined amount of time, a third bar indicating that the user's glucose range is between 70 and 180 mg/dL for 54% of the predefined amount of time, a fourth bar indicating that the user's glucose range is between 54 and 69 mg/dL for 10% of the predefined amount of time, and a fifth bar indicating that the user's glucose range is less than 54 mg/dL for 2% of the predefined amount of time. As with the “Custom” Time-in-Ranges view 305A, those of skill in the art will recognize that the percentages of time associated with each bar can vary depending on the available analyte data of the user. Unlike the “Custom” Time-in-Ranges view 305A, however, the glucose ranges shown in “Standard” view 305B cannot be adjusted by the user.

FIGS. 3C and 3D depict another example embodiment of Time-in-Ranges GUI 320 with multiple views, 320A and 320B, which are analogous to the views shown in FIGS. 3A and 3B, respectively. According to some embodiments, Time-in-Ranges GUI 320 can further include one or more selectable icons 322 (e.g., radio button, check box, slider, switch, etc.) that allow a user to select a predefined amount of time over which the user's analyte data will be shown in the Time-in-Range GUI 320. For example, as shown in FIGS. 3C and 3D, selectable icons 322 can be used to select a predefined amount of time of seven days, fourteen days, thirty days, or ninety days. Those of skill in the art will appreciate that other predefined amounts of time can be utilized and are fully within the scope of this disclosure.

FIG. 3E depicts an example embodiment of a Time-in-Target GUI 330, which can be visually output to a display of a reader device (e.g., a dedicated reader device, a meter device, etc.). In accordance with the disclosed subject matter, Time-in-Target GUI 330 includes three bars comprising (from top to bottom): a first bar indicating that the user's glucose range is above a predefined target range for 34% of a predefined amount of time, a second bar indicating that the user's glucose range is within the predefined target range for 54% of the predefined amount of time, and a third bar indicating that the user's glucose range is below the predefined target range for 12% of the predefined amount of time. Those of skill in the art will recognize that the percentages of time associated with each bar can vary depending on the available analyte data of the user. Furthermore, although FIG. 3E shows a predefined amount of time 332 equal to the last seven days and a predefined target range 334 of 80 to 140 mg/dL, those of skill in the art will appreciate that other predefined amounts of time (e.g., one day, three days, fourteen days, thirty days, ninety days, etc.) and/or predefined target ranges (e.g., 70 to 180 mg/dL) can be utilized, and are fully within the scope of this disclosure.

FIG. 3F depicts another example embodiment of a Time-in-Ranges GUI 340, which includes a single bar comprising five bar portions including (from top to bottom): a first bar portion indicating that the user's glucose range is “Very High” or above 250 mg/dL for 1% (14 minutes) of a predefined amount of time, a second bar portion indicating that the user's glucose range is “High” or between 180 and 250 mg/dL for 18% (4 hours and 19 minutes) of the predefined amount of time, a third bar portion indicating that the user's glucose range is within a “Target Range” or between 70 and 180 mg/dL for 78% (18 hours and 43 minutes) of the predefined amount of time, a fourth bar portion indicating that the user's glucose range is “Low” or between 54 and 69 mg/dL for 3% (43 minutes) of the predefined amount of time, and a fifth bar portion indicating that the user's glucose range is “Very Low” or less than 54 mg/dL for 0% (0 minutes) of the predefined amount of time. As shown in FIG. 3F, according to some embodiments, Time-in-Ranges GUI 340 can display text adjacent to each bar portion indicating an actual amount of time, e.g., in hours and/or minutes.

According to one aspect of the embodiment shown in FIG. 3F, each bar portion of Time-in-Ranges GUI 340 can comprise a different color. In some embodiments, bar portions can be separated by dashed or dotted lines 342 and/or interlineated with numeric markers 344 to indicate the ranges reflected by the adjacent bar portions. In some embodiments, the time in ranges reflected by the bar portions can be further expressed as a percentage, an actual amount of time (e.g., 4 hours and 19 minutes), or, as shown in FIG. 3F, both. Furthermore, those of skill in the art will recognize that the percentages of time associated with each bar portion can vary depending on the analyte data of the user. In some embodiments of Time-in-Ranges GUI 340, the Target Range can be configured by the user. In other embodiments, the Target Range of Time-in-Ranges GUI 340 is not modifiable by the user.

### Example Embodiments of Analyte Level and Trend Alert Interfaces

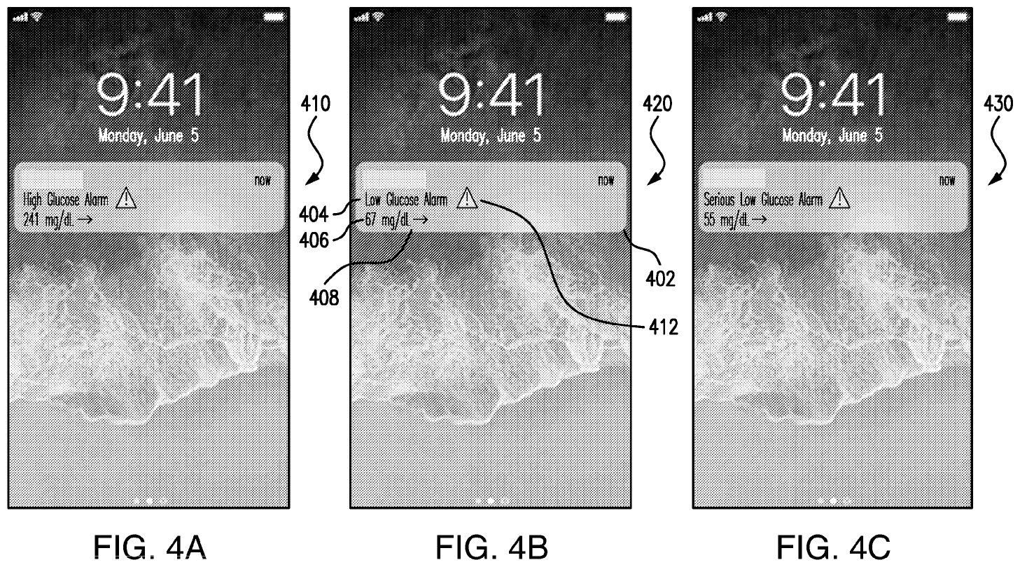

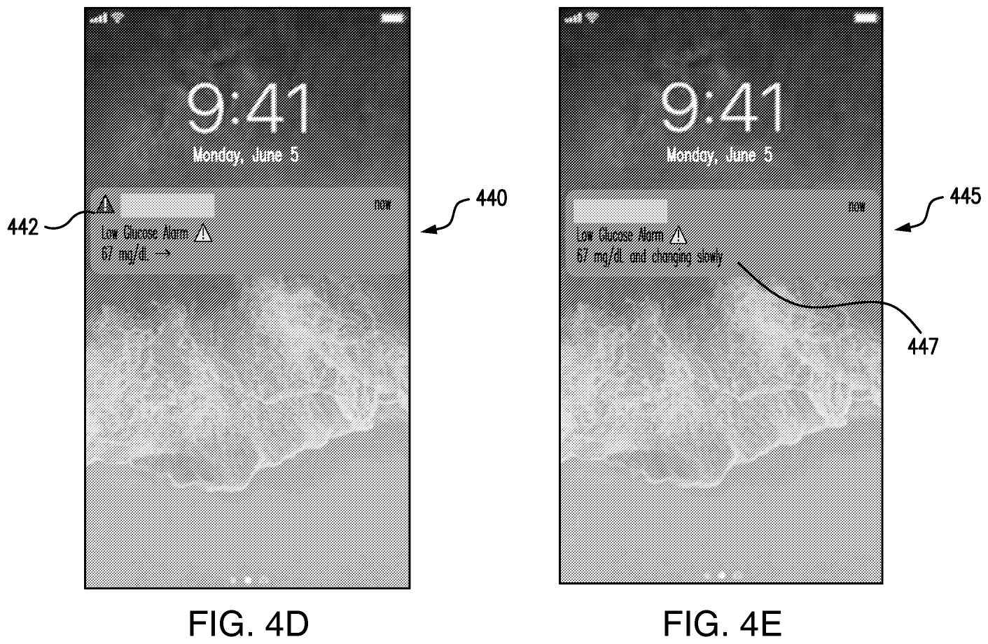

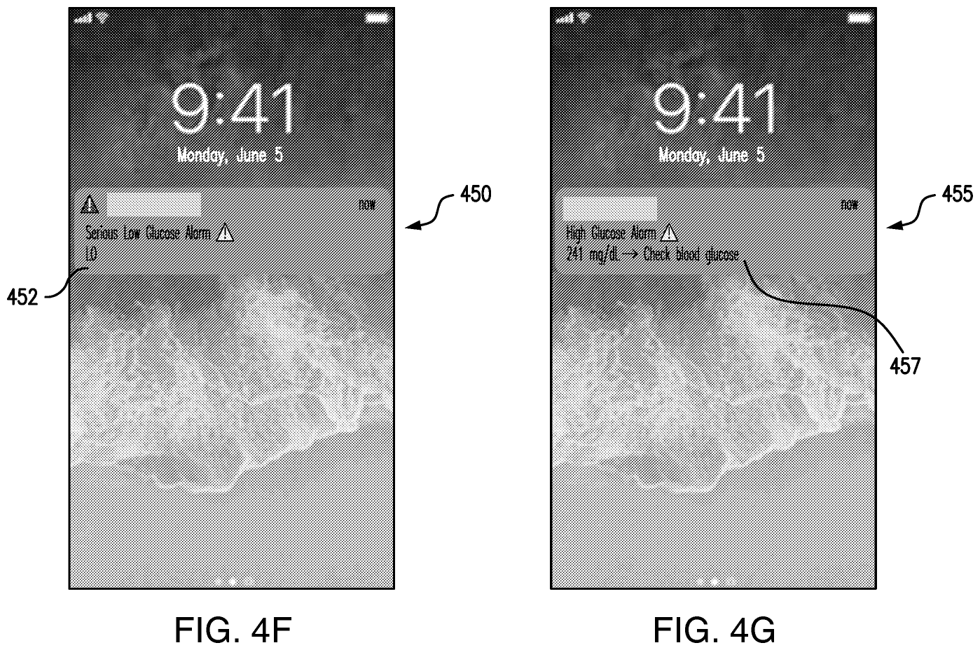

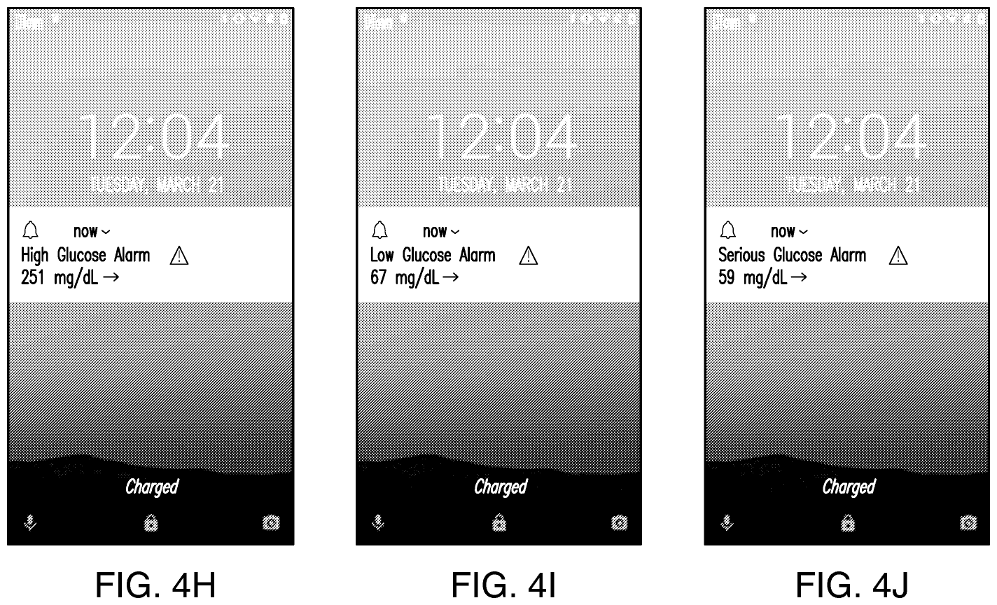

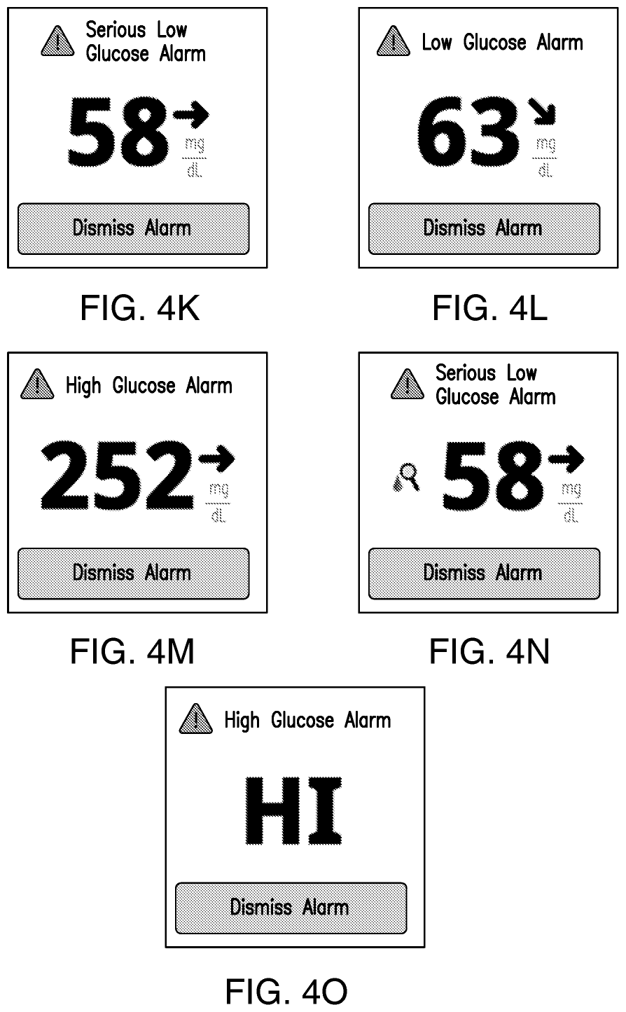

FIGS. 4A to 4O depict example embodiments of Analyte Level/Trend Alert GUIs for analyte monitoring systems. In accordance with the disclosed subject matter, the Analyte Level/Trend Alert GUIs comprise a visual notification (e.g., alert, alarm, pop-up window, banner notification, etc.), wherein the visual notification includes an alarm condition, an analyte level measurement associated with the alarm condition, and a trend indicator associated with the alarm condition.

Turning to FIGS. 4A to 4C, example embodiments of a High Glucose Alarm 410, Low Glucose Alarm 420, and a Serious Low Glucose Alarms 430 are depicted, respectively, wherein each alarm comprises a pop-up window 402 containing an alarm condition text 404 (e.g., “Low Glucose Alarm”), an analyte level measurement 406 (e.g., a current glucose level of 67 mg/dL) associated with the alarm condition, and a trend indicator 408 (e.g., a trend arrow or directional arrow) associated with the alarm condition. In some embodiments, an alarm icon 412 can be adjacent to the alarm condition text 404.

Referring next to FIGS. 4D to 4G, additional example embodiments of Low Glucose Alarms 440, 445, Serious Low Glucose Alarm 450, and High Glucose Alarm 455 are depicted, respectively. As shown in FIG. 4D, Low Glucose Alarm 440 is similar to the Low Glucose Alarm of FIG. 4B (e.g., comprises a pop-up window containing an alarm condition text, an analyte level measurement associated with the alarm condition, and a trend indicator associated with the alarm condition), but further includes an alert icon 442 to indicate that the alarm has been configured as an alert (e.g., will display, play a sound, vibrate, even if the device is locked or if the device's “Do Not Disturb” setting has been enabled). With respect to FIG. 4E, Low Glucose Alarm 445 is also similar to the Low Glucose Alarm of FIG. 4B, but instead of including a trend arrow, Log Glucose Alarm 445 includes a textual trend indicator 447. According to one aspect of some embodiments, textual trend indicator 447 can be enabled through a device's Accessibility settings such that the device will “read” the textual trend indicator 447 to the user via the device's text-to-speech feature (e.g., Voiceover for iOS or Select-to-Speak for Android).

Referring next to FIG. 4F, Low Glucose Alarm 450 is similar to the Low Glucose Alarm of FIG. 4D (including the alert icon), but instead of displaying an analyte level measurement associated with an alarm condition and a trend indicator associated with the alarm condition, Low Glucose Alarm 450 displays a out-of-range indicator 452 to indicate that the current glucose level is either above or below a predetermined reportable analyte level range (e.g., “HI” or “LO”). With respect to FIG. 4G, High Glucose Alarm 455 is similar to the High Glucose Alarm of FIG. 4A (e.g., comprises a pop-up window containing an alarm condition text, an analyte level measurement associated with the alarm condition, and a trend indicator associated with the alarm condition), but further includes an instruction to the user 457. In some embodiments, for example, the instruction can be a prompt for the user to “Check blood glucose.” Those of skill in the art will appreciate that other instructions or prompts can be implemented (e.g., administer a corrective bolus, eat a meal, etc.).

Furthermore, although FIGS. 4A to 4G depict example embodiments of Analyte Level/Trend Alert GUIs that are displayed on smart phones having an iOS operating system, those of skill in the art will also appreciate that the Analyte Level/Trend Alert GUIs can be implemented on other devices including, e.g., smart phones with other operating systems, smart watches, wearables, reader devices, tablet computing devices, blood glucose meters, laptops, desktops, and workstations, to name a few. FIGS. 4H to 4J, for example, depict example embodiments of a High Glucose Alarm, Low Glucose Alarm, and a Serious Low Glucose Alarm for a smart phone having an Android Operating System. Similarly, FIGS. 4K to 4O depict, respectively, example embodiments of a Serious Low Glucose Alarm, Low Glucose Alarm, High Glucose Alarm, Serious Low Glucose Alarm (with a Check Blood Glucose icon), and High Glucose Alarm (with an out-of-range indicator) for a reader device.

### Example Embodiments of Sensor Usage Interfaces

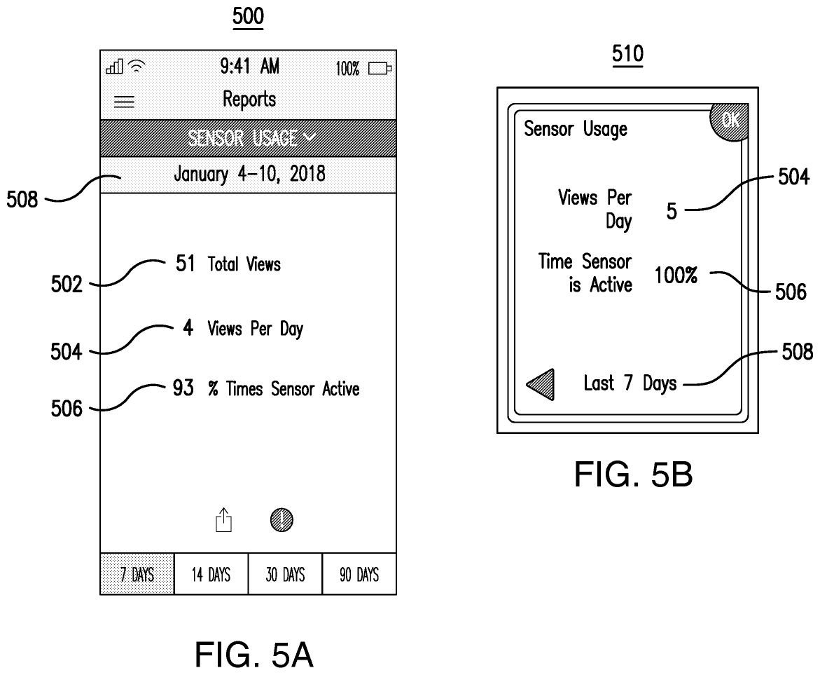

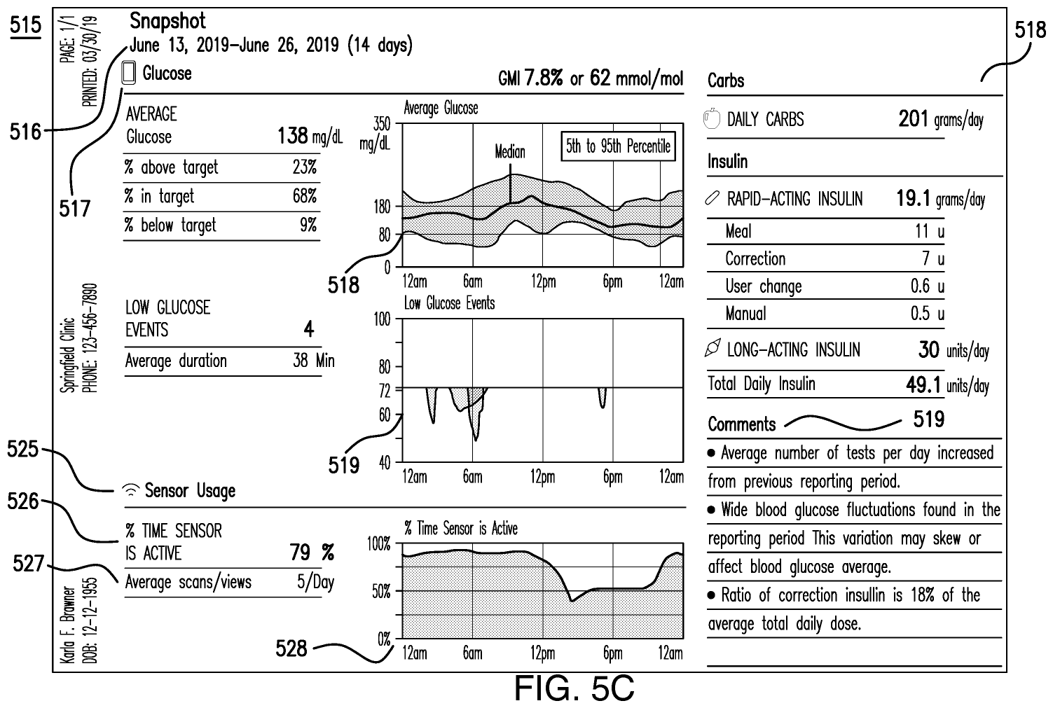

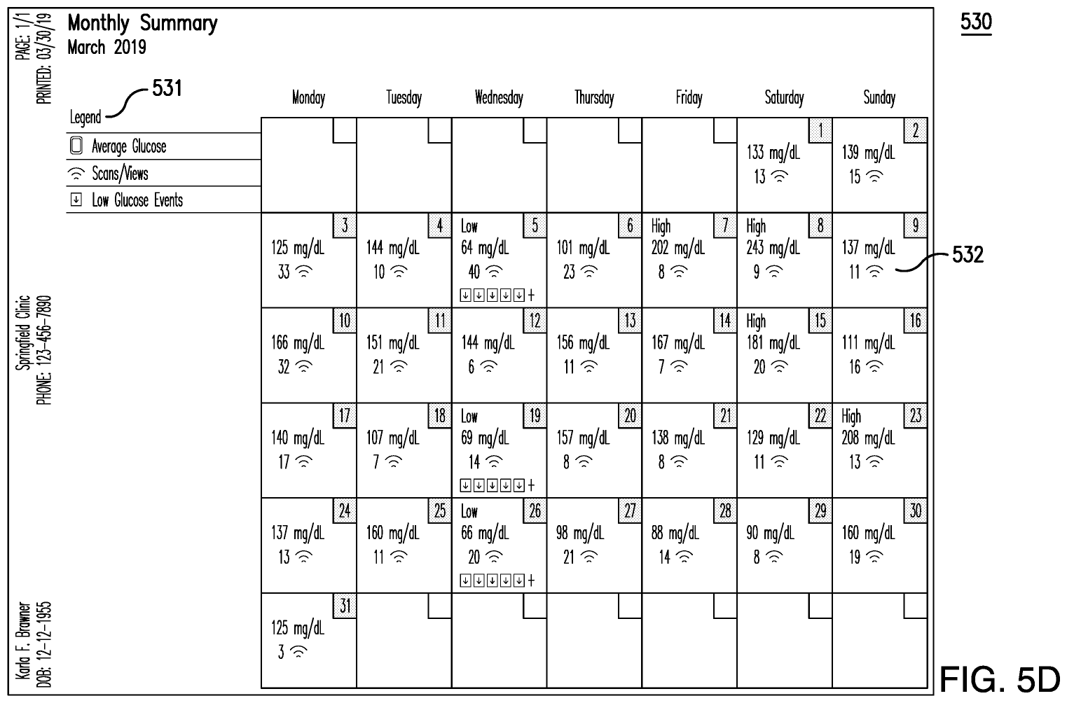

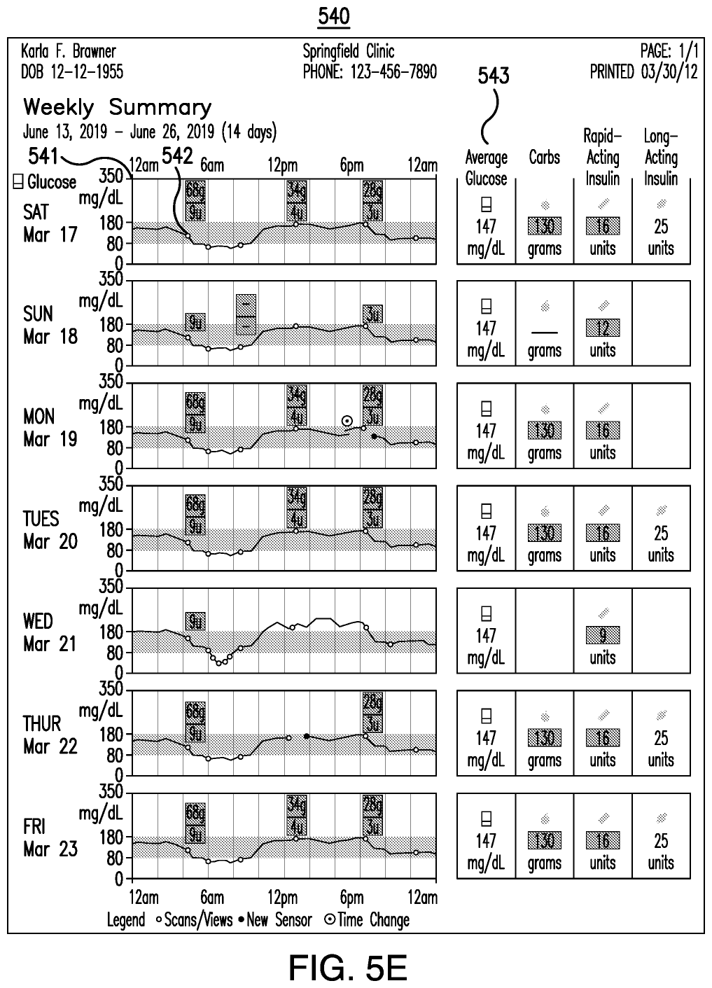

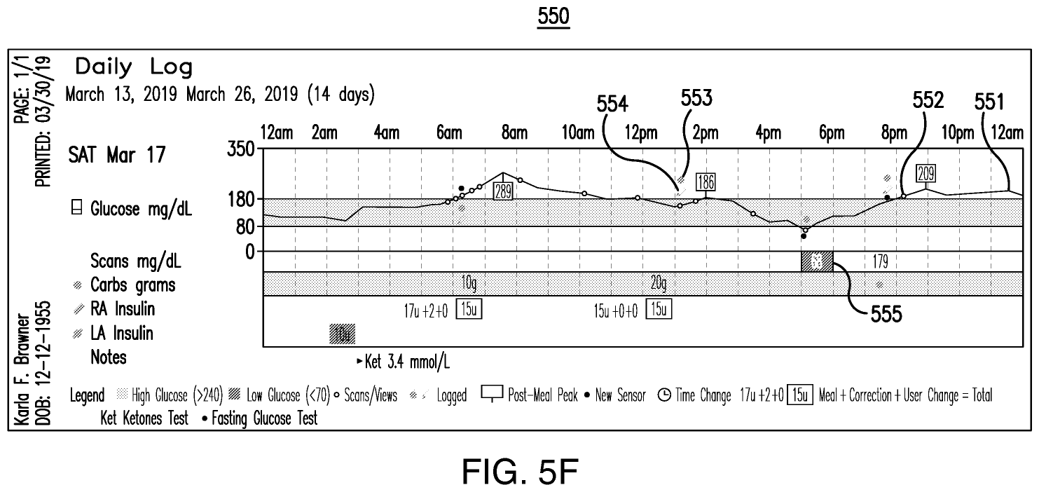

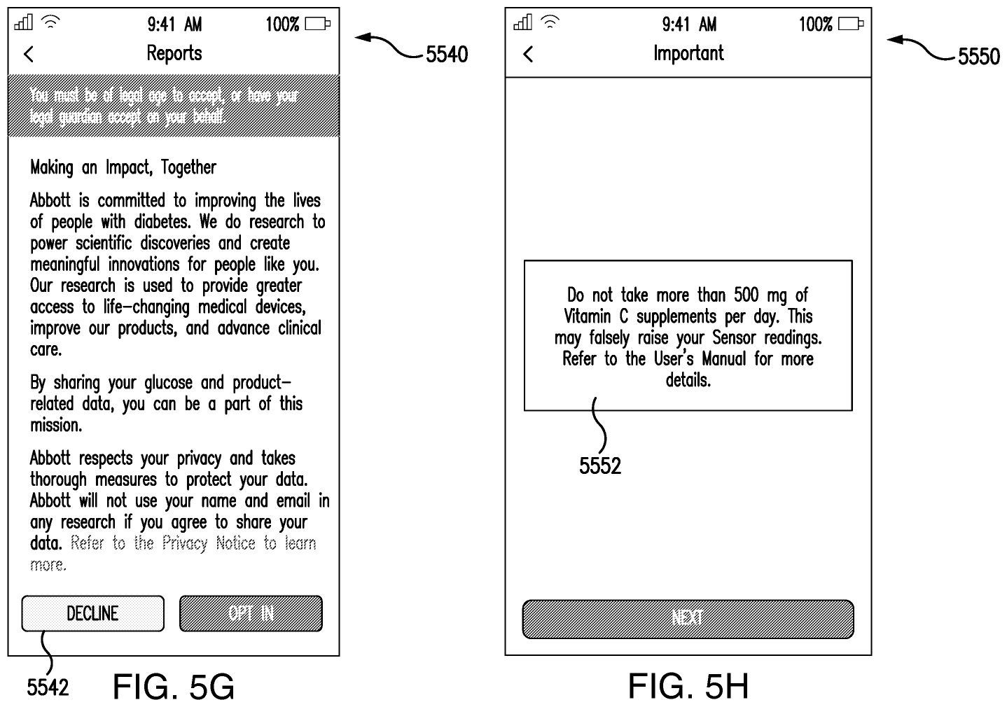

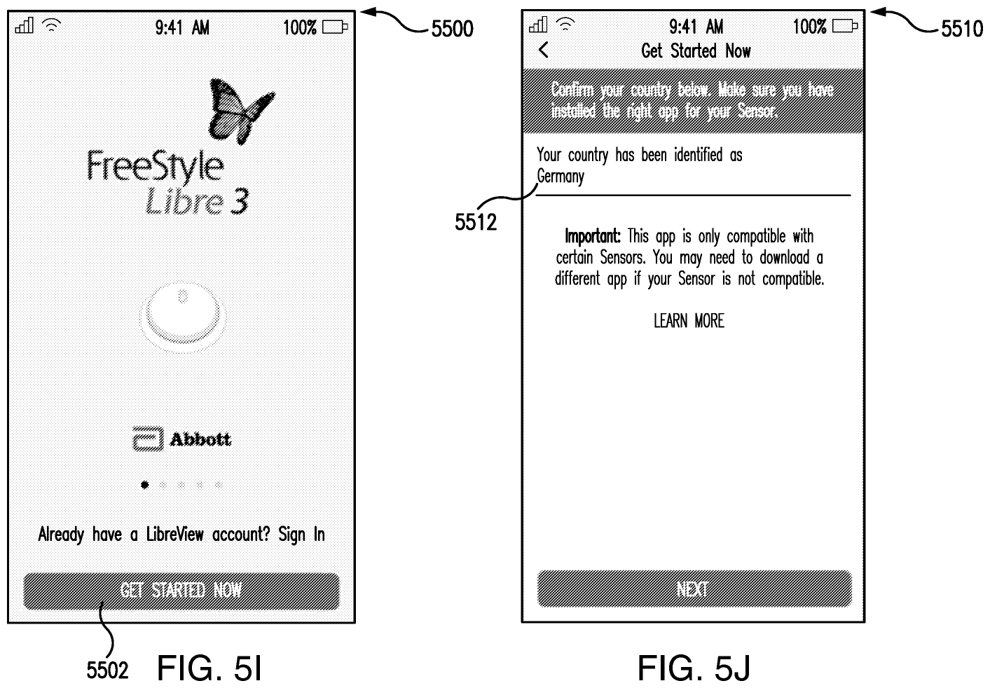

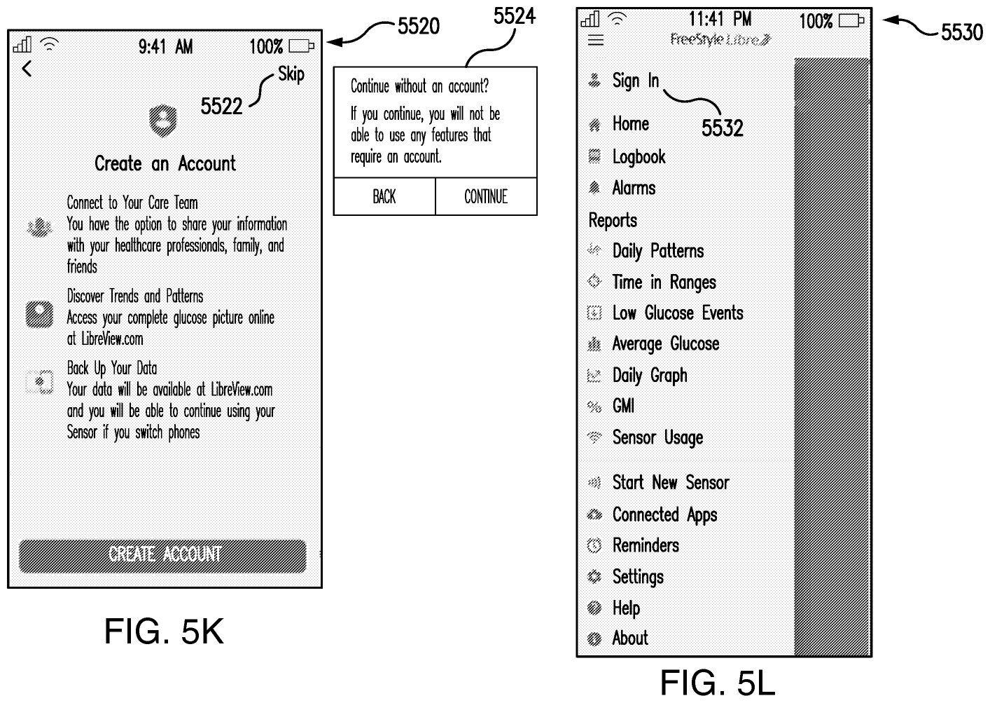

FIGS. 5A to 5F depict example embodiments of sensor usage interfaces relating to GUIs for analyte monitoring systems. In accordance with the disclosed subject matter, sensor usage interfaces provide for technological improvements including the capability to quantify and promote user engagement with analyte monitoring systems. For example, the user can benefit from subtle behavioral modification as the sensor usage interface encourages more frequent interaction with the device and the expected improvement in outcomes. The user can also benefit from increased frequent interaction which leads to improvement in a number of metabolic parameters, as discussed in further detail below.

In some embodiments, HCPs can receive a report of the user's frequency of interaction and a history of the patient's recorded metabolic parameters (e.g., estimated HbA1c levels, time in range of 70-180 mg/dL, etc.). If an HCP sees certain patients in their practice are less engaged than others, the HCPs can focus their efforts on improving engagement in users/patients that are less engaged than others. HCPs can benefit from more cumulative statistics (such as average glucose views per day, average glucose views before/after meals, average glucose views on “in-control” vs. “out-of-control” days or time of day) which may be obtained from the record of user's interaction frequency with the analyte monitoring systems and which can be used to understand why a patient may not be realizing expected gains from the analyte monitoring system. If an HCP sees that a patient is not benefiting as expected from the analyte monitoring system, they may recommend an increased level of interaction (e.g., increase interaction target level). Accordingly, an HCP can change the predetermined target level of interaction.

In some embodiments, caregivers can receive a report of the user's frequency of interaction. In turn, caregivers may be able to nudge the user to improve interaction with the analyte monitoring system. The caregivers may be able to use the data to better understand and improve their level of engagement with the user's analyte monitoring systems or alter therapy decisions.

According to some embodiments, for example, a sensor usage interface can include the visual display of one or more “view” metrics, each of which can be indicative of a measure of user engagement or interaction with the analyte monitoring system. A “view” can comprise, for example, an instance in which a sensor results interface is rendered or brought into the foreground (e.g., in certain embodiments, to view any of the GUI described herein). In some embodiments, the update interval as described above, data on sensor results GUI 245 is automatically updated or refreshed according to an update interval (e.g., every second, every minute, every 5 minutes, etc.). As such, a “view” can comprise one instance per update interval in which a sensor results interface is rendered or brought into the foreground. For example, if the update interval is every minute, rendering or bringing into the foreground the sensor results GUI 245 several times in that minute would only comprise one “view.” Similarly, if the sensor results GUI 245 is rendered or brought into the foreground for 20 continuous minutes, data on the senor results GUI 245 would be updated 20 times (i.e., once every minute). However, this would only constitute 20 “views” (i.e., one “view” per update interval). Similarly, if the update interval is every five minutes, rendering or bringing into the foreground the sensor results GUI 245 several times in those five minutes would only comprise one “view.” If the sensor results interface is rendered or brought into the foreground for 20 continuous minutes, this would constitute 4 “views” (i.e., one “view” each for each of the four five-minute intervals). According to other embodiments, a “view” can be defined as an instance when a user views a sensor results interface with a valid sensor reading for the first time in a sensor lifecount. According to disclosed embodiments, user can receive a notification, as described below, indicating when an instance of rendering or brining into the foreground the sensor results GUI is not counted as a “view.” For example, the user can receive a visual notification indicating such as “Results have not updated,” or “View does not count,” or “Please check glucose level again.” In some embodiments, the user can receive a check-in for each instance which counts as a “view,” as described in greater detail below.

According to disclosed embodiments, the one or more processors can be configured to record no more than one instance of user operation of the reader device during a defined time period. For example, and not limitation, a defined time period can include an hour. A person of ordinary skill in the art would understand defined time period to include any appropriate period of time, such as, one hour, two hours, three hours, 30 minutes, 15 minutes, etc.

According to some embodiments, a “view” can comprise, for example, a visual notification (e.g., prompt, alert, alarm, pop-up window, banner notification, etc.). In some embodiments, the visual notification can include an alarm condition, an analyte level measurement associated with the alarm condition, and a trend indicator associated with the alarm condition. For example, Analyte Level/Trend Alert GUIs, such as those embodiments depicted in FIGS. 4A to 40 can constitute a “view.”

In some embodiments, a sensor user interface can include a visual display of a “scan” metric indicative of another measure of user engagement or interaction with the analyte monitoring system. A “scan” can comprise, for example, an instance in which a user uses a reader device (e.g., smart phone, dedicated reader, etc.) to scan a sensor control device, such as, for example, in a Flash Analyte Monitoring system. As described above in connection with “views”, a “scan” can comprise one instance per update interval in a user uses a reader device to scan a sensor control device.

FIG. 5A and 5B depict example embodiments of sensor usage interfaces 500 and 510, respectively. In accordance with the disclosed subject matter, sensor usage interfaces 500 and 510 can be rendered and displayed, for example, by a mobile app or software residing in non-transitory memory of reader device 120, such as those described with respect to FIGS. 1 and 2A. In some embodiments, for each instance of a “views” or “scans,” the software can record the date and time of the user's interaction with the system. In some embodiments, for each instance of a “view” or “scan,” the software can record the current glucose value. Referring to FIG. 5A, sensor user interface 500 can comprise: a predetermined time period interval 508 indicative of a time period (e.g., a date range) during which view metrics are measured, a Total Views metric 502, which is indicative of a total number of views over the predetermined time period 508; a Views Per Day metric 504, which is indicative of an average number of views per day over the predetermined time period 508; and a Percentage Time Sensor Active metric 506, which is indicative of the percentage of predetermined time period 508 that reader device 120 is in communication with sensor control device 102, such as those described with respect to FIGS. 1, 2B, and 2C. Referring to FIG. 5B, sensor user interface 510 can comprise a Views per Day metric 504 and a Percentage Time Sensor Active metric 508, each of which is measured for predetermined time period 508.

According to another aspect of the embodiments, although predetermined time period 508 is shown as one week, those of skill in the art will recognize that other predetermined time periods (e.g., 3 days, 14 days, 30 days) can be utilized. In addition, predetermined time period 508 can be a discrete period of time—with a start date and an end date—as shown in sensor usage interface 500 of FIG. 5A, or can be a time period relative to a current day or time (e.g., “Last 7 Days,” “Last 14 Days,” etc.), as shown in sensor usage interface 510 of FIG. 5B.

FIG. 5C depicts an example embodiment of sensor usage interface 525, as part of analyte monitoring system report GUI 515. In accordance with the disclosed subject matter, GUI 515 is a snapshot report covering a predetermined time period 516 (e.g., 14 days), and comprising a plurality of report portions on a single report GUI, including: a sensor usage interface portion 525, a glucose trend interface 517, which can include an glucose trend graph, a low glucose events graph, and other related glucose metrics (e.g., Glucose Management Indicator); a health information interface 518, which can include information logged by the user about the user's average daily carbohydrate intake and medication dosages (e.g., insulin dosages); and a comments interface 519, which can include additional information about the user's analyte and medication patterns presented in a narrative format. According to another aspect of the embodiments, sensor usage interface 525 can comprise a Percentage Time Sensor Active metric 526, an Average Scans/Views metric 527 (e.g., indicative of an average sum of a number of scans and a number of views), and a Percentage Time Sensor Active graph 528. As can be seen in FIG. 5C, an axis of the Percentage Time Sensor Active graph can be aligned with a corresponding axis of one or more other graphs (e.g., average glucose trend graph, low glucose events graph), such that the user can visually correlate data between multiple graphs from two or more portions of the report GUI by the common units (e.g., time of day) from the aligned axes.

FIG. 5D depicts an example embodiment of another analyte monitoring system report GUI 530 including sensor usage information. In accordance with the disclosed subject matter, GUI 530 is a monthly summary report including a first portion comprising a legend 531, wherein legend 531 includes a plurality of graphical icons each of which is adjacent to a descriptive text. As shown in FIG. 5D, legend 531 includes an icon and descriptive text for “Average Glucose,” an icon and descriptive text for “Scans/Views,” and an icon and descriptive text for “Low Glucose Events.” GUI 530 also includes a second portion comprising a calendar interface 532. For example, as shown in FIG. 5D, GUI 530 comprises a monthly calendar interface, wherein each day of the month can include one or more of an average glucose metric, low glucose event icons, and a sensor usage metric 532. In some embodiments, such as the one shown in FIG. 5D, the sensor usage metric (“scans/views”) is indicative of a total sum of a number of scans and a number of views for each day.

FIG. 5E depicts an example embodiment of another analyte monitoring system report GUI 540 including sensor usage information. In accordance with the disclosed subject matter, GUI 540 is a weekly summary report including a plurality of report portions, wherein each report portion is representative of a different day of the week, and wherein each report portion comprises a glucose trend graph 541, which can include the user's measured glucose levels over a twenty-four hour period, and a health information interface 543, which can include information about the user's average daily glucose, carbohydrate intake, and/or insulin dosages. In some embodiments, glucose trend graph 541 can include sensor usage markers 542 to indicate that a scan, a view, or both had occurred at a particular time during the twenty-four hour period.

FIG. 5F depicts an example embodiment of another analyte monitoring system report GUI 550 including sensor usage information. In accordance with the disclosed subject matter, GUI 550 is a daily log report comprising a glucose trend graph 551, which can include the user's glucose levels over a twenty-four hour period. In some embodiments, glucose trend graph 551 can include sensor usage markers 552 to indicate that a scan, a view, or both had occurred at a particular time during the twenty-four hour period. Glucose trend graph 551 can also include logged event markers, such as logged carbohydrate intake markers 553 and logged insulin dosage markers 554, as well as glucose event markers, such as low glucose event markers 555.

FIGS. 5I to 5L depict various GUIs for improving usability and user privacy with respect to analyte monitoring software. FIG. 5G, GUI 5540 depicts a research consent interface 5540, which prompts the user to choose to either decline or opt in (through buttons 5542) with respect to permitting the user's analyte data and/or other product-related data to be used for research purposes. According to embodiments of the disclosed subject matter, the analyte data can be anonymized (de-identified) and stored in an international database for research purposes.

Referring next to FIG. 5H, GUI 5550 depicts a “Vitamin C” warning interface 5550 which displays a warning to the user that the daily use of more than 500 mg of Vitamin C supplements can result in falsely high sensor readings.

FIG. 5I is GUI 5500 depicting a first start interface which can be displayed to a user the first time the analyte monitoring software is started. In accordance with the disclosed subject matter, GUI 5500 can include a “Get Started Now” button 5502 that, when pressed, will navigate the user to GUI 5510 of FIG. 5J. GUI 5510 depicts a country confirmation interface 5512 that prompts the user to confirm the user's country. According to another aspect of the embodiments, the country selected can limit and/or enable certain interfaces within the analyte monitoring software application for regulatory compliance purposes.

Turning next to FIG. 5K, GUI 5520 depicts a user account creation interface which allows the user to initiate a process to create a cloud-based user account. In accordance with the disclosed subject matter, a cloud-based user account can allow the user to share information with healthcare professionals, family and friends; utilize a cloud-based reporting platform to review more sophisticated analyte reports; and back up the user's historical sensor readings to a cloud-based server. In some embodiments, GUI 5520 can also include a “Skip” link 5522 that allows a user to utilize the analyte monitoring software application in an “accountless mode” (e.g., without creating or linking to a cloud-based account). Upon selecting the “Skip” link 5522, an information window 5524 can be displayed to inform that certain features are not available in “accountless mode.” Information window 5524 can further prompt the user to return to GUI 5520 or proceed without account creation.

FIG. 5L is GUI 5530 depicting a menu interface displayed within an analyte monitoring software application while the user is in “accountless mode.” According to an aspect of the embodiments, GUI 5530 includes a “Sign in” link 5532 that allows the user to leave “accountless mode” and either create a cloud-based user account or sign-in with an existing cloud-based user account from within the analyte monitoring software application.

It will be understood by those of skill in the art that any of the GUIs, reports interfaces, or portions thereof, as described herein, are meant to be illustrative only, and that the individual elements, or any combination of elements, depicted and/or described for a particular embodiment or figure are freely combinable with any elements, or any combination of elements, depicted and/or described with respect to any of the other embodiments.

### Example Embodiments of Digital Interfaces for Analyte Monitoring Systems

Described herein are example embodiments of digital interfaces for analyte monitoring systems. In accordance with the disclosed subject matter, a digital interface can comprise a series of instructions, routines, subroutines, and/or algorithms, such as software and/or firmware stored in a non-transitory memory, executed by one or more processors of one or more devices in an analyte monitoring system, wherein the instructions, routines, subroutines, or algorithms are configured to enable certain functions and inter-device communications. As an initial matter, it will be understood by those of skill in the art that the digital interfaces described herein can comprise instructions stored in a non-transitory memory of a sensor control device 102, reader device 120, local computer system 170, trusted computer system 180, and/or any other device or system that is part of, or in communication with, analyte monitoring system 100, as described with respect to FIGS. 1, 2A, and 2B. These instructions, when executed by one or more processors of the sensor control device 102, reader device 120, local computer system 170, trusted computer system 180, or other device or system of analyte monitoring system 100, cause the one or more processors to perform the method steps described herein. Those of skill in the art will further recognize that the digital interfaces described herein can be stored as instructions in the memory of a single centralized device or, in the alternative, can be distributed across multiple discrete devices in geographically dispersed locations.

### Example Embodiments of Methods for Data Backfilling

Example embodiments of methods for data backfilling in an analyte monitoring system will now be described. In accordance with the disclosed subject matter, gaps in analyte data and other information can result from interruptions to communication links between various devices in an analyte monitoring system 100. These interruptions can occur, for example, from a device being powered off (e.g., a user's smart phone runs out of battery), or a first device temporarily moving out of a wireless communication range from a second device (e.g., a user wearing sensor control device 102 inadvertently leaves her smart phone at home when she goes to work). As a result of these interruptions, reader device 120 may not receive analyte data and other information from sensor control device 102. It would thus be beneficial to have a robust and flexible method for data backfilling in an analyte monitoring system to ensure that once a communication link is re-established, each analyte monitoring device can receive a complete set of data, as intended.

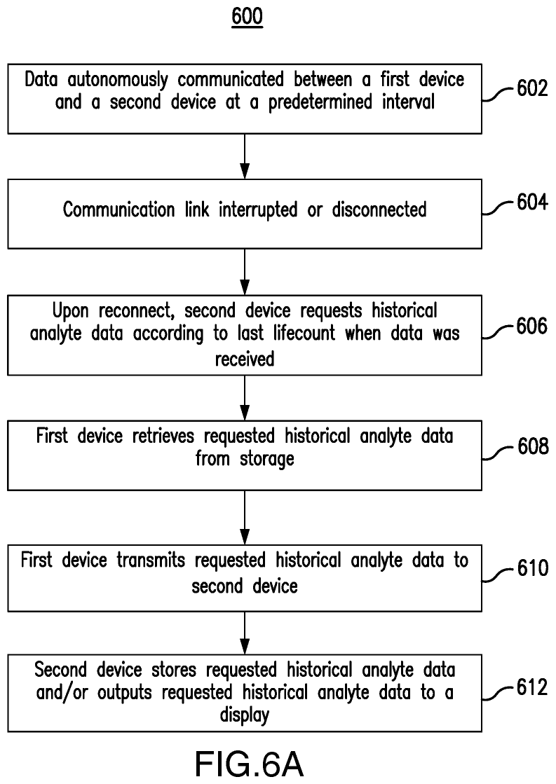

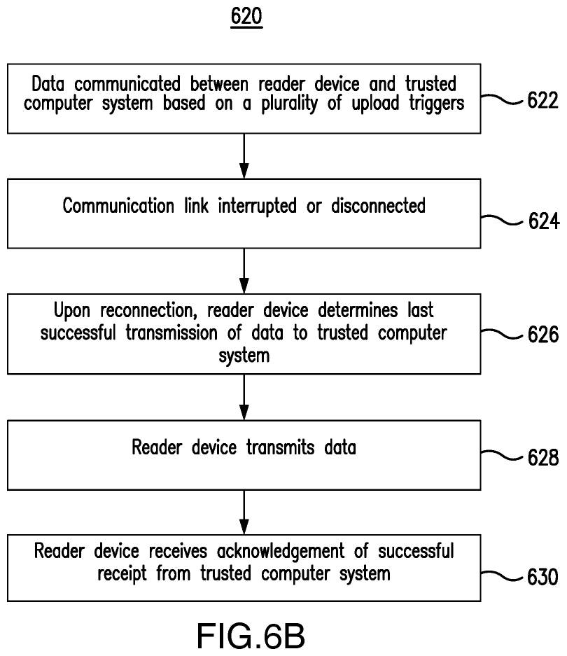

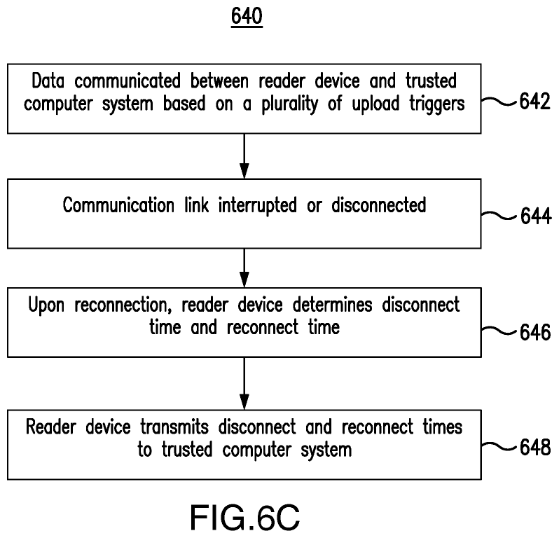

FIG. 6A is a flow diagram depicting an example embodiment of a method 600 for data backfilling in an analyte monitoring system. In accordance with the disclosed subject matter, method 600 can be implemented to provide data backfilling between a sensor control device 102 and a reader device 120. At Step 602, analyte data and other information is autonomously communicated between a first device and a second device at a predetermined interval. In some embodiments, the first device can be a sensor control device 102, and the second device can be a reader device 120, as described with respect to FIGS. 1, 2A, and 2B. In accordance with the disclosed subject matter, analyte data and other information can include, but is not limited to, one or more of: data indicative of an analyte level in a bodily fluid, a rate-of-change of an analyte level, a predicted analyte level, a low or a high analyte level alert condition, a sensor fault condition, or a communication link event. According to another aspect of the embodiments, autonomous communications at a predetermined interval can comprise streaming analyte data and other information according to a standard wireless communication network protocol, such as a Bluetooth or Bluetooth Low Energy protocol, at one or more predetermined rates (e.g., every minute, every five minutes, every fifteen minutes, etc.). In some embodiments, different types of analyte data or other information can be autonomously communicated between the first and second devices at different predetermined rates (e.g., historical glucose data every 5 minutes, current glucose value every minute, etc.).

At Step 604, a disconnection event or condition occurs that causes an interruption to the communication link between the first device and the second device. As described above, the disconnection event can result from the second device (e.g., reader device 120, smart phone, etc.) running out of battery power or being powered off manually by a user. A disconnection event can also result from the first device being moved outside a wireless communication range of the second device, from the presence of a physical barrier that obstructs the first device and/or the second device, or from anything that otherwise prevents wireless communications from occurring between the first and second devices.

At Step 606, the communication link is re-established between the first device and the second device (e.g., the first device comes back into the wireless communication range of the second device). Upon reconnection, the second device requests historical analyte data according to a last lifecount metric for which data was received. In accordance with the disclosed subject matter, the lifecount metric can be a numeric value that is incremented and tracked on the second device in units of time (e.g., minutes), and is indicative of an amount of time elapsed since the sensor control device was activated. For example, in some embodiments, after the second device (e.g., reader device 120, smart phone, etc.) re-establishes a Bluetooth wireless communication link with the first device (e.g., sensor control device 120), the second device can determine the last lifecount metric for which data was received. Then, according to some embodiments, the second device can send to the first device a request for historical analyte data and other information having a lifecount metric greater than the determined last lifecount metric for which data was received.

In some embodiments, the second device can send a request to the first device for historical analyte data or other information associated with a specific lifecount range, instead of requesting historical analyte data associated with a lifecount metric greater than a determined last lifecount metric for which data was received.

At Step 608, upon receiving the request, the first device retrieves the requested historical analyte data from storage (e.g., non-transitory memory of sensor control device 102), and subsequently transmits the requested historical analyte data to the second device at Step 610. At Step 612, upon receiving the requested historical analyte data, the second device stores the requested historical analyte data in storage (e.g., non-transitory memory of reader device 120). In accordance with the disclosed subject matter, when the requested historical analyte data is stored by the second device, it can be stored along with the associated lifecount metric. In some embodiments, the second device can also output the requested historical analyte data to a display of the second device, such as, for example to a glucose trend graph of a sensor results GUI, such as those described with respect to FIGS. 2D to 2I. For example, in some embodiments, the requested historical analyte data can be used to fill in gaps in a glucose trend graph by displaying the requested historical analyte data along with previously received analyte data.

Furthermore, those of skill in the art will appreciate that the method of data backfilling can be implemented between multiple and various devices in an analyte monitoring system, wherein the devices are in wired or wireless communication with each other.

FIG. 6B is a flow diagram depicting another example embodiment of a method 620 for data backfilling in an analyte monitoring system. In accordance with the disclosed subject matter, method 620 can be implemented to provide data backfilling between a reader device 120 (e.g., smart phone, dedicated reader) and a trusted computer system 180, such as, for example, a cloud-based platform for generating reports. At Step 622, analyte data and other information is communicated between reader device 120 and trusted computer system 180 based on a plurality of upload triggers. In accordance with the disclosed subject matter, analyte data and other information can include, but are not limited to, one or more of: data indicative of an analyte level in a bodily fluid (e.g., current glucose level, historical glucose data), a rate-of-change of an analyte level, a predicted analyte level, a low or a high analyte level alert condition, information logged by the user, information relating to sensor control device 102, alarm information (e.g., alarm settings), wireless connection events, and reader device settings, to name a few.

According to another aspect of the embodiments, the plurality of upload triggers can include (but is not limited to) one or more of the following: activation of sensor control device 102; user entry or deletion of a note or log entry; a wireless communication link (e.g., Bluetooth) reestablished between reader device 120 and sensor control device 102; alarm threshold changed; alarm presentation, update, or dismissal; internet connection re-established; reader device 120 restarted; a receipt of one or more current glucose readings from sensor control device 102; sensor control device 120 terminated; signal loss alarm presentation, update, or dismissal; signal loss alarm is toggled on/off; view of sensor results screen GUI; or user sign-in into cloud-based platform.

According to another aspect of the embodiments, in order to track the transmission and receipt of data between devices, reader device 120 can “mark” analyte data and other information that is to be transmitted to trusted computer system 180. In some embodiments, for example, upon receipt of the analyte data and other information, trusted computer system 180 can send a return response to reader device 120, to acknowledge that the analyte data and other information has been successfully received. Subsequently, reader device 120 can mark the data as successfully sent. In some embodiments, the analyte data and other information can be marked by reader device 120 both prior to being sent and after receipt of the return response. In other embodiments, the analyte data and other information can be marked by reader device 120 only after receipt of the return response from trusted computer system 180.

Referring to FIG. 6B, at Step 624, a disconnection event occurs that causes an interruption to the communication link between reader device 120 and trusted computer system 180. For example, the disconnection event can result from the user placing the reader device 120 into “airplane mode” (e.g., disabling of the wireless communication modules), from the user powering off the reader device 120, or from the reader device 120 moving outside of a wireless communication range.

At Step 626, the communication link between reader device 120 and trusted computer system 180 (as well as the internet) is re-established, which is one of the plurality of upload triggers. Subsequently, reader device 120 determines the last successful transmission of data to trusted computer system 180 based on the previously marked analyte data and other information sent. Then, at Step 628, reader device 120 can transmit analyte data and other information not yet received by trusted computer system 180. At Step 630, reader device 120 receives acknowledgement of successful receipt of analyte data and other information from trusted computer system 180.

Although FIG. 6B is described above with respect to a reader in communication with a trusted computer system, those of skill in the art will appreciate that the data backfilling method can be applied between other devices and computer systems in an analyte monitoring system (e.g., between a reader and a local computer system, between a reader and a medical delivery device, between a reader and a wearable computing device, etc.). These embodiments, along with their variations and permutations, are fully within the scope of this disclosure.

In addition to data backfilling, example embodiments of methods for aggregating disconnect and reconnect events for wireless communication links in an analyte monitoring system are described. In accordance with the disclosed subject matter, there can be numerous and wide-ranging causes for interruptions to wireless communication links between various devices in an analyte monitoring system. Some causes can be technical in nature (e.g., a reader device is outside a sensor control device's wireless communication range), while other causes can relate to user behavior (e.g., a user leaving his or her reader device at home). In order to improve connectivity and data integrity in analyte monitoring systems, it would therefore be beneficial to gather information regarding the disconnect and reconnect events between various devices in an analyte monitoring system.

FIG. 6C is a flow diagram depicting an example embodiment of a method 640 for aggregating disconnect and reconnect events for wireless communication links in an analyte monitoring system. In some embodiments, for example, method 640 can be used to detect, log, and upload to trusted computer system 180, Bluetooth or Bluetooth Low Energy disconnect and reconnect events between a sensor control device 102 and a reader device 120. In accordance with the disclosed subject matter, trusted computer system 180 can aggregate disconnect and reconnect events transmitted from a plurality of analyte monitoring systems. The aggregated data can then by analyzed to determine whether any conclusions can be made about how to improve connectivity and data integrity in analyte monitoring systems.

At Step 642, analyte data and other information are communicated between reader device 120 and trusted computer system 180 based on a plurality of upload triggers, such as those previously described with respect to method 620 of FIG. 6B. At Step 644, a disconnection event occurs that causes an interruption to the wireless communication link between sensor control device 102 and reader device 120. Example disconnection events can include, but are not limited to, a user placing the reader device 120 into “airplane mode,” the user powering off the reader device 120, the reader device 120 running out of power, the sensor control device 102 moving outside a wireless communication range of the reader devices 120, or a physical barrier obstructing the sensor control device 102 and/or the reader device 120, to name only a few.

Referring still to FIG. 6C, at Step 646, the wireless communication link between the sensor control device 102 and reader device 120 is re-established, which is one of the plurality of upload triggers. Subsequently, reader device 120 determines a disconnect time and a reconnect time, wherein the disconnect time is the time that the interruption to the wireless communication link began, and the reconnect time is the time that the wireless communication link between the sensor control device 102 and reader device 120 is re-established. According to some embodiments, the disconnection and reconnection times can also be stored locally in an event log on reader device 120. At Step 648, reader device 120 transmits the disconnect and reconnect times to trusted computer system 180.

According to some embodiments, the disconnect and reconnect times can be stored in non-transitory memory of trusted computer system 180, such as in a database, and aggregated with the disconnect and reconnect times collected from other analyte monitoring systems. In some embodiments, the disconnect and reconnect times can also be transmitted to and stored on a different cloud-based platform or server from trusted computer system 180 that stores analyte data. In still other embodiments, the disconnect and reconnect times can be anonymized.

In addition, those of skill in the art will recognize that method 640 can be utilized to collect disconnect and reconnect times between other devices in an analyte monitoring system, including, for example: between reader device 120 and trusted computer system 180; between reader device 120 and a wearable computing device (e.g., smart watch, smart glasses); between reader device 120 and a medication delivery device (e.g., insulin pump, insulin pen); between sensor control device 102 and a wearable computing device; between sensor control device 102 and a medication delivery device; and any other combination of devices within an analyte monitoring system. Those of skill in the art will further appreciate that method 640 can be utilized to analyze disconnect and reconnect times for different wireless communication protocols, such as, for example, Bluetooth or Bluetooth Low Energy, NFC, 802.11x, UHF, cellular connectivity, or any other standard or proprietary wireless communication protocol.

### Example Embodiments of Improved Expired Failed Sensor Transmissions

Example embodiments of methods for improved expired and/or failed sensor transmissions in an analyte monitoring system will now be described. In accordance with the disclosed subject matter, expired or failed sensor conditions detected by a sensor control device 102 can trigger alerts on reader device 120. However, if the reader device 120 is in “airplane mode,” powered off, outside a wireless communication range of sensor control device 102, or otherwise unable to wirelessly communicate with the sensor control device 102, then the reader device 120 may not receive these alerts. This can cause the user to miss information such as, for example, the need to promptly replace a sensor control device 102. Failure to take action on a detected sensor fault can also lead to the user being unaware of adverse glucose conditions (e.g., hypoglycemia and/or hyperglycemia) due to a terminated sensor.

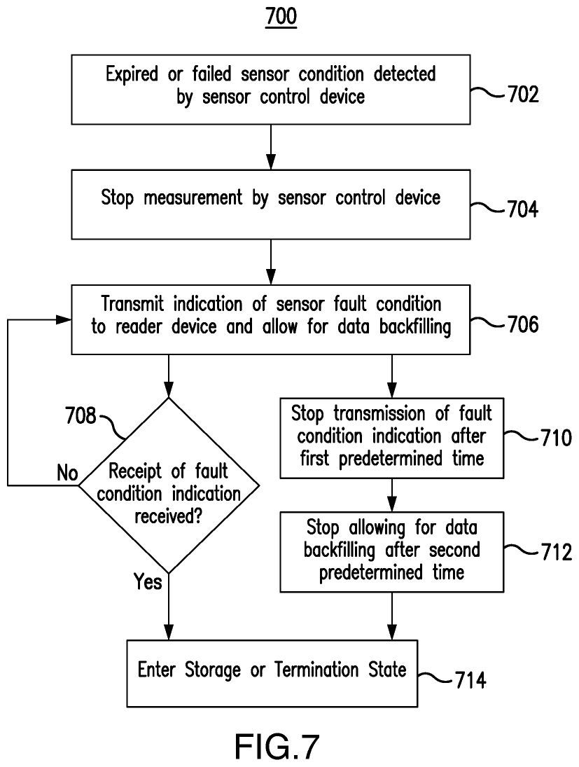

FIG. 7 is a flow diagram depicting an example embodiment of a method 700 for improved expired or failed sensor transmissions in an analyte monitoring system. In accordance with the disclosed subject matter, method 700 can be implemented to provide for improved sensor transmissions by a sensor control device 102 after an expired or failed sensor condition has been detected. At Step 702, an expired or failed sensor condition is detected by sensor control device 102. In some embodiments, the sensor fault condition can comprise one or both of a sensor insertion failure condition or a sensor termination condition. According to some embodiments, for example, a sensor insertion failure condition or a sensor termination condition can include, but is not limited to, one or more of the following: a FIFO overflow condition detected, a sensor signal below a predetermined insertion failure threshold, moisture ingress detected, an electrode voltage exceeding a predetermined diagnostic voltage threshold, an early signal attenuation (ESA) condition, or a late signal attenuation (LSA) condition, to name a few.

Referring again to FIG. 7, at Step 704, sensor control device 102 stops acquiring measurements of analyte levels from the analyte sensor in response to the detection of the sensor fault condition. At Step 706, sensor control device 102 begins transmitting an indication of a sensor fault condition to reader device 120, while also allowing for the reader device 120 to connect to the sensor control device 102 for purposes of data backfilling. In accordance with the disclosed subject matter, the transmission of the indication of the sensor fault condition can comprise transmitting a plurality of Bluetooth or Bluetooth Low Energy advertising packets, each of which can include the indication of the sensor fault condition. In some embodiments, the plurality of Bluetooth or BLE advertising packets can be transmitted repeatedly, continuously, or intermittently. Those of skill in the art will recognize that other modes of wirelessly broadcasting or multicasting the indication of the sensor fault condition can be implemented. According to another aspect of the embodiments, in response to receiving the indication of the sensor fault condition, reader device 120 can visually display an alert or prompt for a confirmation by the user.

At Step 708, sensor control device 102 can be configured to monitor for a return response or acknowledgment of receipt of the indication of the sensor fault condition from reader device 120. In some embodiments, for example, a return response or acknowledgement of receipt can be generated by reader device 120 when a user dismisses an alert on the reader device 120 relating to the indication of the sensor fault condition, or otherwise responds to a prompt for confirmation of the indication of the sensor fault condition. If a return response or acknowledgement of receipt of the indication of the sensor fault condition is received by sensor control device 102, then at Step 714, sensor control device 102 can enter either a storage state or a termination state. According to some embodiments, in the storage state, the sensor control device 102 is placed in a low-power mode, and the sensor control device 102 is capable of being re-activated by a reader device 120. By contrast, in the termination state, the sensor control device 102 cannot be re-activated and must be removed and replaced.

If a receipt of the fault condition indication is not received by sensor control device 102, then at Step 710, the sensor control device 102 will stop transmitting the fault condition indication after a first predetermined time period. In some embodiments, for example, the first predetermined time period can be one of: one hour, two hours, five hours, etc. Subsequently, at Step 712, if a receipt of the fault condition indication is still not received by sensor control device 102, then at Step 712, the sensor control device 102 will also stop allowing for data backfilling after a second predetermined time period. In some embodiments, for example, the second predetermined time period can be one of: twenty-four hours, forty-eight hours, etc. Sensor control device 102 then enters a storage state or a termination state at Step 714.

By allowing sensor control device 102 to continue transmissions of sensor fault conditions for a predetermined time period, the embodiments of this disclosure mitigate the risk of unreceived sensor fault alerts. In addition, although the embodiments described above are in reference to a sensor control device 102 in communication with a reader device 120, those of skill in the art will recognize that indications of sensor fault conditions can also be transmitted between a sensor control device 102 and other types of mobile computing devices, such as, for example, wearable computing devices (e.g., smart watches, smart glasses) or tablet computing devices.

### Example Embodiments of Data Merging in Analyte Monitoring Systems

Example embodiments of methods for merging data received from one or more analyte monitoring systems will now be described. As described earlier with respect to FIG. 1, a trusted computer system 180, such as a cloud-based platform, can be configured to generate various reports based on received analyte data and other information from a plurality of reader devices 120 and sensor control devices 102. A large and diverse population of reader devices and sensor control devices, however, can give rise to complexities and challenges in generating reports based on the received analyte data and other information. For example, a single user may have multiple reader devices and/or sensor control devices, either simultaneously or serially over time, each of which can comprise different versions. This can lead to further complications in that, for each user, there may be sets of duplicative and/or overlapping data. It would therefore be beneficial to have methods for merging data at a trusted computer system for purposes of report generation.

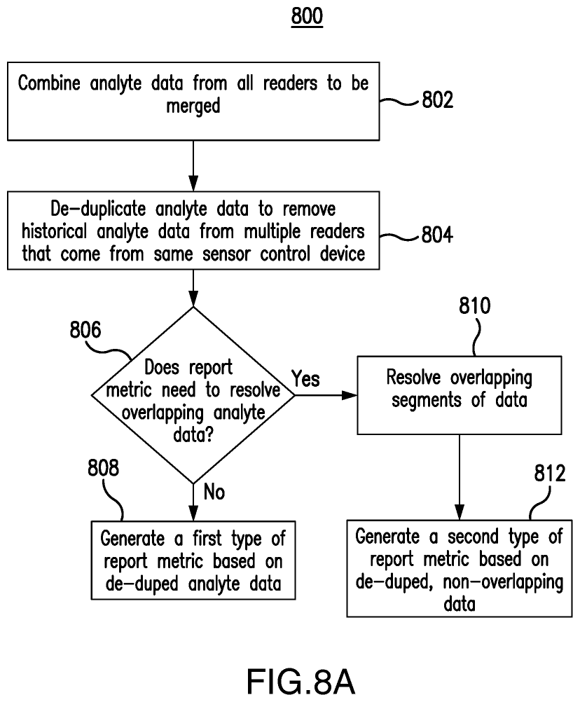

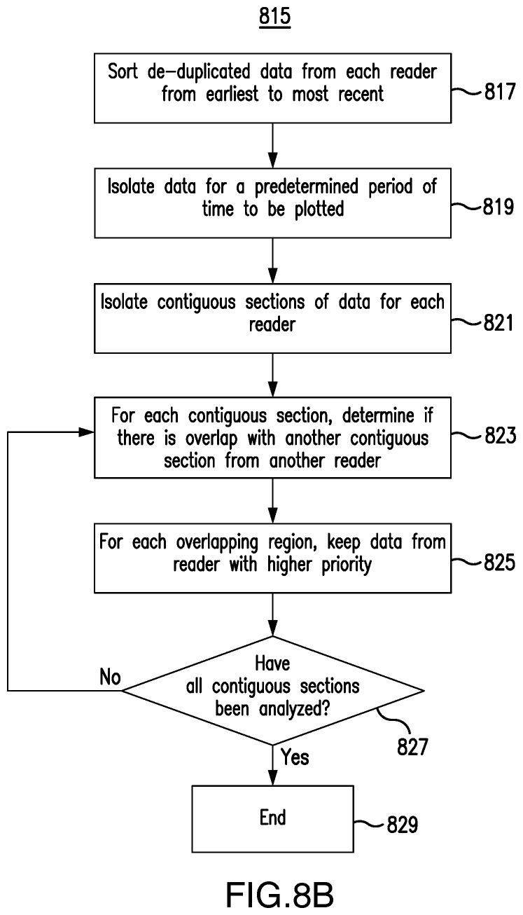

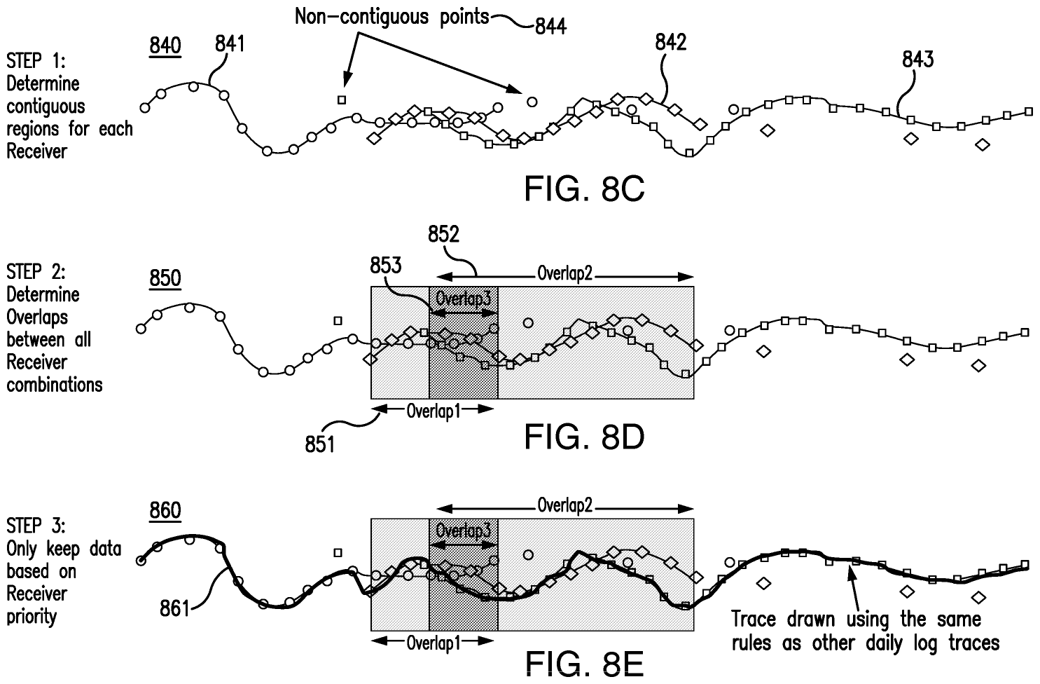

FIG. 8A is a flow diagram depicting an example embodiment of a method 800 for merging data associated with a user and generating one or more report metrics, wherein the data originates from multiple reader devices and multiple sensor control devices. In accordance with the disclosed subject matter, method 800 can be implemented to merge analyte data in order to generate different types of report metrics utilized in various reports. At Step 802, data is received from one or more reader devices 120 and combined for purposes of merging. At Step 804, the combined data is then de-duplicated to remove historical data from multiple readers originating from the same sensor control device. In accordance with the disclosed subject matter, the process of de-duplicating data can include (1) identifying or assigning a priority associated with each reader device from which analyte data is received, and (2) in the case where there is “duplicate” data, preserving the data associated with the reader device with a higher priority. In some embodiments, for example, a newer reader device (e.g., newer model, having a more recent version of software installed) is assigned a higher priority than an older reader device (e.g., older model, having an older version of software installed). In some embodiments, priority can be assigned by device type (e.g., smart phone having a higher priority over a dedicated reader).

Referring still to FIG. 8A, at Step 806, a determination is made as to whether one or more of the report metrics to be generated requires resolution of overlapping data. If not, at Step 808, a first type of report metric can be generated based on de-duplicated data without further processing. In some embodiments, for example, the first type of report metric can include average glucose levels used in reports, such as a snapshot or monthly summary report (as described with respect to FIGS. 5C and 5D). If it is determined that one or more of the report metrics to be generated requires resolution of overlapping data, then at Step 810, a method for resolving overlapping regions of data is performed. An example embodiment method for resolving overlapping regions of data is described below with respect to FIG. 8B. Subsequently, at Step 812, a second type of report metric based on data that has been de-duplicated and processed to resolve overlapping data segments, is generated. In some embodiments, for example, the second type of report metric can include low glucose event calculations used in reports, such as the daily log report (as described with respect to FIG. 5F).

FIG. 8B is a flow diagram depicting an example embodiment of a method 815 for resolving overlapping regions of analyte data, which can be implemented, for example, in Step 810 of method 800, as described with respect to FIG. 8A. At Step 817, the de-duplicated data from each reader (resulting from Step 804 of method 800, as described with respect to FIG. 8A) can be sorted from earliest to most recent. At Step 819, based on the report metric to be generated, the de-duplicated and sorted data is then isolated according to a predetermined period of time. In some embodiments, for example, if the report metric is a graph reflecting glucose values over a specific day, then the de-duplicated and sorted data can be isolated for that specific day. Next, at Step 821, contiguous sections of the de-duplicated and sorted data for each reader device are isolated. In accordance with the disclosed subject matter, non-contiguous data points can be discarded or disregarded (e.g., not used) for purposes of generating report metrics. At Step 823, for each contiguous section of de-duplicated and sorted data of a reader device, a determination is made as to whether there are any overlapping regions with other contiguous sections of de-duplicated and sorted data from other reader devices. At Step 825, for each overlapping region identified, the de-duplicated and sorted data from the reader device with the higher priority is preserved. At Step 827, if it is determined that all contiguous sections have been analyzed according to the previous steps, then method 815 ends at Step 829. Otherwise, method 815 then returns to Step 823 to continue identifying and resolving any overlapping regions between contiguous sections of de-duplicated and sorted data for different reader devices.

FIGS. 8C to 8E are graphs (840, 850, 860) depicting various stages of de-duplicated and sorted data from multiple reader devices, as the data is processed according to method 815 for resolving overlapping regions of data. Referring first to FIG. 8C, graph 840 depicts de-duplicated and sorted data from three different reader devices: a first reader 841 (as reflected by the circular data points), a second reader 842 (as reflected by diamond-shaped data points), and a third reader 843 (as reflected by the square-shaped data points). According to one aspect of graph 840, the data is depicted at Step 821 of method 815, after it has been de-duplicated, sorted, and isolated to a predetermined time period. As can be seen in FIG. 8C, a contiguous section of data for each of the three reader devices (841, 842, and 843) has been identified, and three traces are shown. According to another aspect of the graph 840, non-contiguous points 844 are not included in the three traces.

Referring next to FIG. 8D, graph 850 depicts the data from readers 841, 842, 843 at Step 823 of method 815, wherein three overlapping regions between the contiguous sections of data have been identified: a first overlapping region 851 between all three contiguous sections of data; a second overlapping region 852 between two contiguous sections of data (from reader device 842 and reader device 843); and a third overlapping region 853 between two contiguous sections of data (also from reader device 842 and reader device 843).

FIG. 8E is a graph 860 depicting data at Step 825 of method 815, wherein a single trace 861 indicates the merged, de-duplicated, and sorted data from three reader devices 841, 842, 843 after overlapping regions 851, 852, and 853 have been resolved by using the priority of each reader device. According to graph 860, the order of priority from highest to lowest is: reader device 843, reader device 842, and reader device 841.

Although FIGS. 8C, 8D, and 8E depict three contiguous sections of data with three discrete overlapping regions identified, those of skill in the art will understand that either fewer or more contiguous sections of data (and non-contiguous data points) and overlapping regions are possible. For example, those of skill in the art will recognize that where a user has only two reader devices, there may be fewer contiguous sections of data and overlapping regions, if any at all. Conversely, if a user has five reader devices, those of skill in the art will understand that there may be five contiguous sections of data with three or more overlapping regions.

### Example Embodiments of Sensor Transitioning

Example embodiments of methods for sensor transitioning will now be described. In accordance with the disclosed subject matter, as mobile computing and wearable technologies continue to advance at a rapid pace and become more ubiquitous, users are more likely to replace or upgrade their smart phones more frequently. In the context of analyte monitoring systems, it would therefore be beneficial to have sensor transitioning methods to allow a user to continue using a previously activated sensor control device with a new smart phone. In addition, it would also be beneficial to ensure that historical analyte data from the sensor control device could be backfilled to the new smart phone (and subsequently uploaded to the trusted computer system) in a user-friendly and secure manner.

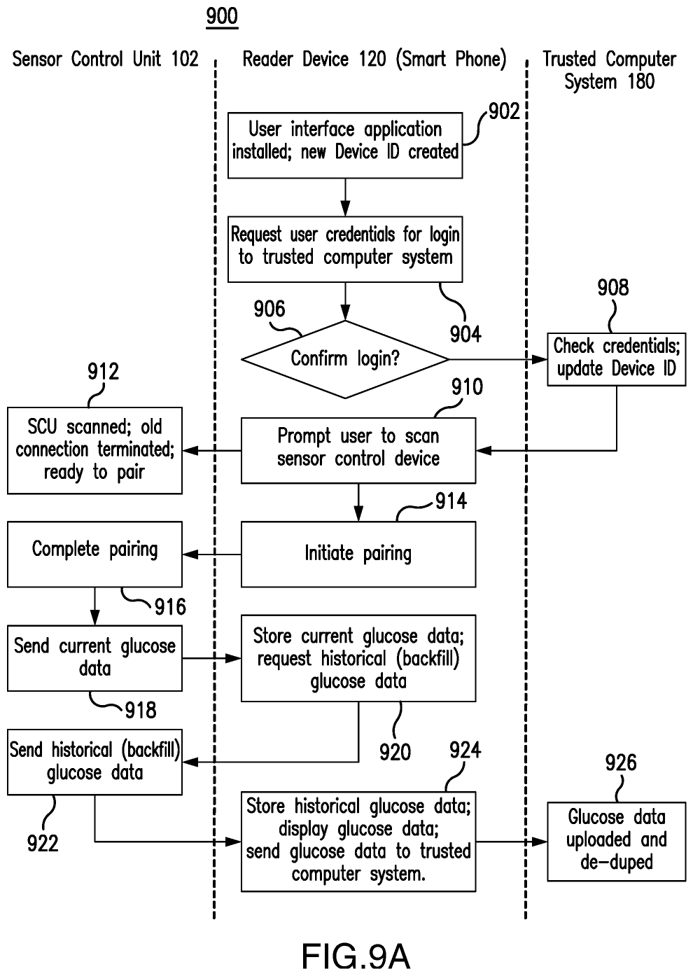

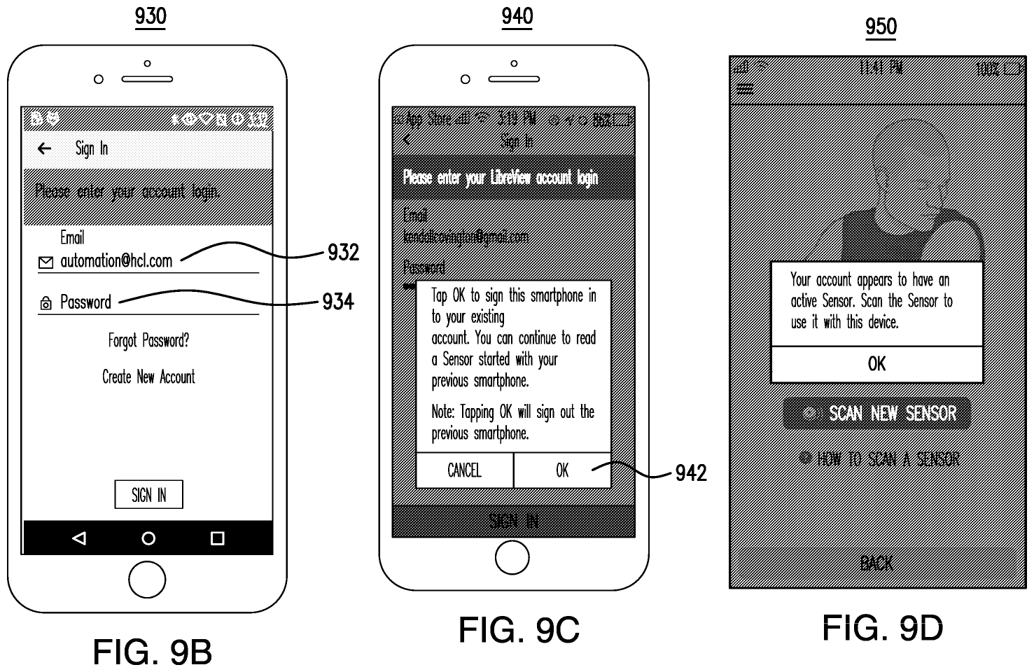

FIG. 9A is a flow diagram depicting an example embodiment of a method 900 for transitioning a sensor control device. In accordance with the disclosed subject matter, method 900 can be implemented in an analyte monitoring system to allow a user to continue using a previously activated sensor control device with a new reader device (e.g., smart phone). At Step 902, a user interface application (e.g., mobile software application or app) is installed on reader device 120 (e.g., smart phone), which causes a new unique device identifier, or “device ID,” to be created and stored on reader device 120. At Step 904, after installing and launching the app, the user is prompted to enter their user credentials for purposes of logging into trusted computer system 180 (e.g., cloud-based platform or server). An example embodiment of a GUI 930 for prompting the user to enter their user credentials is shown in FIG. 9B. According to an aspect of the embodiments, GUI 930 can include a username field 932, which can comprise a unique username or an e-mail address, and a masked or unmasked password field 934, to allow the user to enter their password.

Referring again to FIG. 9A, at Step 906, after user credentials are entered into the app, a prompt is displayed requesting user confirmation to login to trusted computer system 180. An example embodiment of GUI 940 for requesting user confirmation to login to trusted computer system 180 is shown in FIG. 9D. According to an aspect of the embodiments, GUI 940 can also include a warning, such as the one shown in FIG. 9D, that confirming the login will cause the user to be logged off from other reader devices (e.g., the user's old smart phone).

If the user confirms login, then at Step 908, the user's credentials are sent to trusted computer system 180 and subsequently verified. In addition, according to some embodiments, the device ID can also be transmitted from the reader device 120 to trusted computer system 180 and stored in a non-transitory memory of trusted computer system 180. According to some embodiments, for example, in response to receiving the device ID, trusted computer system 180 can update a device ID field associated with the user's record in a database.

After the user credentials are verified by trusted computer system 180, at Step 910, the user is prompted by the app to scan the already-activated sensor control device 102. In accordance with the disclosed subject matter, the scan can comprise bringing the reader device 120 in close proximity to sensor control device 102, and causing the reader device 120 to transmit one or more wireless interrogation signals according to a first wireless communication protocol. In some embodiments, for example, the first wireless communication protocol can be a Near Field Communication (NFC) wireless communication protocol. Those of skill in the art, however, will recognize that other wireless communication protocols can be implemented (e.g., infrared, UHF, 802.11x, etc.). An example embodiment of GUI 950 for prompting the user to scan the already-activated sensor control device 102 is shown in FIG. 9D.

Referring still to FIG. 9A, at Step 912, scanning of sensor control device 102 by reader device 120 causes sensor control device 102 to terminate an existing wireless communication link with the user's previous reader device, if there is currently one established. According to an aspect of the embodiments, the existing wireless communication link can comprise a link established according to a second wireless communication protocol that is different from the first wireless communication protocol. In some embodiments, for example, the second wireless communication protocol can be a Bluetooth or Bluetooth Low Energy protocol. Subsequently, sensor control device 102 enters into a “ready to pair” state, in which sensor control device 102 is available to establish a wireless communication link with reader device 120 according to the second wireless communication protocol.

At Step 914, reader device 120 initiates a pairing sequence via the second wireless communication protocol (e.g., Bluetooth or Bluetooth Low Energy) with sensor control device 102. Subsequently, at Step 916, sensor control device 102 completes the pairing sequence with reader device 120. At Step 918, sensor control device 102 can begin sending current glucose data to reader device 120 according to the second wireless communication protocol. In some embodiments, for example, current glucose data can be wirelessly transmitted to reader device 120 at a predetermined interval (e.g., every minute, every two minutes, every five minutes).

Referring still to FIG. 9A, at Step 920, reader device 120 receives and stores current glucose data received from sensor control device 102 in a non-transitory memory of reader device 120. In addition, according to some embodiments, reader device 120 can request historical glucose data from sensor control device 102 for backfilling purposes. According to some embodiments, for example, reader device 120 can request historical glucose data from sensor control device 102 for the full wear duration, which is stored in a non-transitory memory of sensor control device 102. In other embodiments, reader device 120 can request historical glucose data for a specific predetermined time range (e.g., from day 3 to present, from day 5 to present, last 3 days, last 5 days, lifecount>0, etc.). Those of skill will appreciate that other backfilling schemes can be implemented (such as those described with respect to FIGS. 6A and 6B), and are fully within the scope of this disclosure.

Upon receipt of the request at Step 922, sensor control device 102 can retrieve historical glucose data from a non-transitory memory and transmit it to reader device 120. In turn, at Step 924, reader device 120 can store the received historical glucose data in a non-transitory memory. In addition, according to some embodiments, reader device 120 can also display the current and/or historical glucose data in the app (e.g., on a sensor results screen). In this regard, a new reader can display all available analyte data for the full wear duration of a sensor control device. In some embodiments, reader device 120 can also transmit the current and/or historical glucose data to trusted computer system 180. At Step 926, the received glucose data can be stored in a non-transitory memory (e.g., a database) of trusted computer system 180.

In some embodiments, the received glucose data can also be de-duplicated prior to storage in non-transitory memory.

### Example Embodiments of Check Sensor and Replace Sensor System Alarms

Example embodiments of autonomous check sensor and replace sensor system alarms, and methods relating thereto, will now be described. In accordance with the disclosed subject matter, certain adverse conditions affecting the operation of the analyte sensor and sensor electronics can be detectable by the sensor control device. For example, an improperly inserted analyte sensor can be detected if an average glucose level measurement over a predetermined period of time is determined to be below an insertion failure threshold. Due to its small form factor and a limited power capacity, however, the sensor control device may not have sufficient alarming capabilities. As such, it would be advantageous for the sensor control device to transmit indications of adverse conditions to another device, such as a reader device (e.g., smart phone), to alert the user of those conditions.

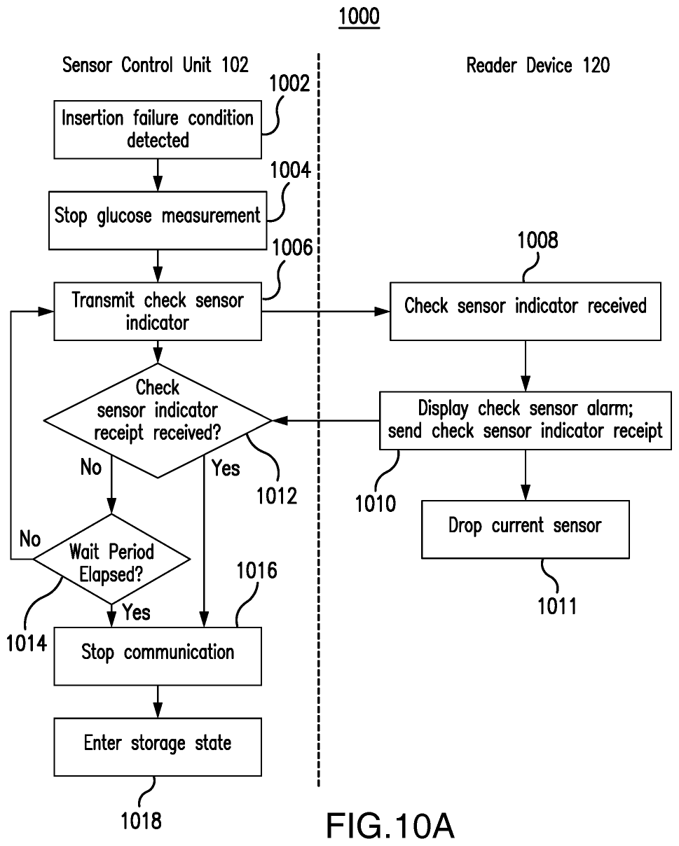

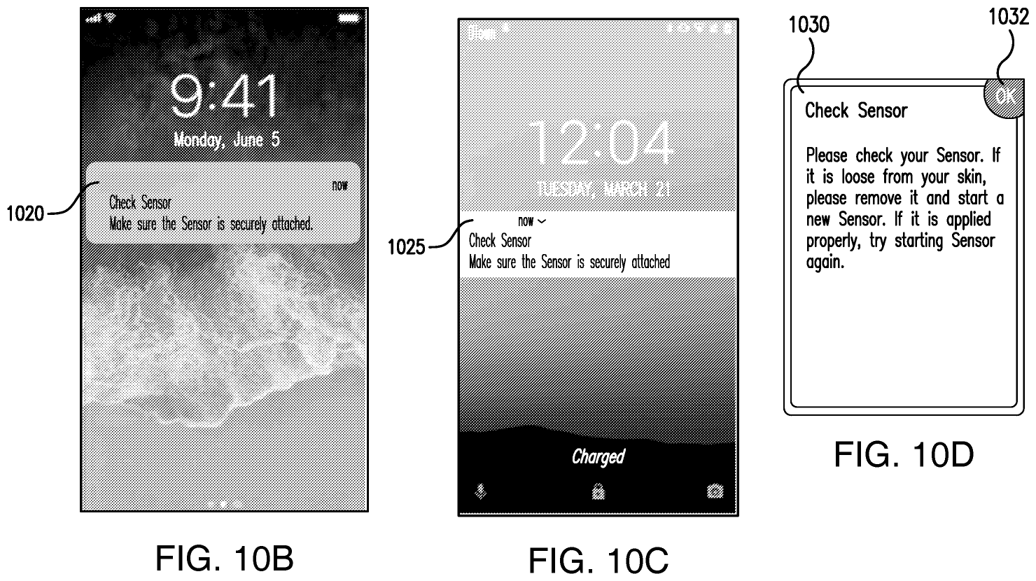

FIG. 10A is a flow diagram depicting an example embodiment of a method 1000 for generating a sensor insertion failure system alarm (also referred to as a “check sensor” system alarm). At Step 1002, a sensor insertion failure condition is detected by sensor control device 102. In some embodiments, for example, a sensor insertion failure condition can be detected when an average glucose value during a predetermined time period (e.g., average glucose value over five minutes, eight minutes, 15 minutes, etc.) is below an insertion failure glucose level threshold. At Step 1004, in response to the detection of the insertion failure condition, sensor control device 102 stops taking glucose measurements. At Step 1006, sensor control device 102 generates a check sensor indicator and transmits it via wireless communication circuitry to reader device 120. Subsequently, as shown at Steps 1012 and 1014, sensor control device 102 will continue to transmit the check sensor indicator until either: (1) a receipt of the indicator is received from reader device 120 (step 1012); or (2) a predetermined waiting period has elapsed (Step 1014), whichever occurs first.

According to another aspect of the embodiments, if a wireless communication link is established between sensor control device 102 and reader device 120, then reader device 120 will receive the check sensor indicator at Step 1008. In response to receiving the check sensor indicator, reader device 120 will display a check sensor system alarm at Step 1010. FIGS. 10B to 10D are example embodiments of check sensor system alarm interfaces, as displayed on reader device 120. In some embodiments, for example, the check sensor system alarm can be a notification box, banner, or pop-up window that is output to a display of a smart phone, such as interfaces 1020 and 1025 of FIGS. 10B and 10C. In some embodiments, the check sensor alarm can be output to a display on a reader device 120, such as a glucose meter or a receiver device, such as interface 1030 of FIG. 10D. According to the embodiments, reader device 120 can also transmit a check sensor indicator receipt back to sensor control device 102. In some embodiments, for example, the check sensor indicator receipt can be automatically generated and sent upon successful display of the check sensor system alarm 1020, 1025, or 1030. In other embodiments, the check sensor indicator receipt is generated and/or transmitted in response to a predetermined user input (e.g., dismissing the check sensor system alarm, pressing a confirmation ‘OK’ button 1032, etc.).

Subsequently, at Step 1011, reader device 120 drops sensor control device 102. In accordance with the disclosed subject matter, for example, Step 1011 can comprise one or more of: terminating an existing wireless communication link with sensor control device 102; unpairing from sensor control device 102; revoking an authorization or digital certificate associated with sensor control device 102; creating or modifying a record stored on reader device 120 to indicate that sensor control device 102 is in a storage state; or transmitting an update to trusted computer system 180 to indicate that sensor control device 102 is in a storage state.

Referring back to FIG. 10A, if either the check sensor indicator receipt is received (at Step 1012) by sensor control device 102 or the predetermined wait period has elapsed (Step 1014), then at Step 1016, sensor control device 102 stops the transmission of check sensor indicators. Subsequently, at Step 1018, sensor control device 102 enters a storage state in which sensor control device 102 does not take glucose measurements and the wireless communication circuitry is either de-activated or transitioned into a dormant mode. According to one aspect, while in a ‘storage state,’ sensor control device 102 can be re-activated by reader device 120.

Although method 1000 of FIG. 10A is described with respect to glucose measurements, those of skill in the art will appreciate that sensor control device 102 can be configured to measure other analytes (e.g., lactate, ketone, etc.) as well. In addition, although method 1000 of FIG. 10A describes certain method steps performed by reader device 120 (e.g., receiving check sensor indicator, displaying a check sensor system alarm, and sending a check sensor indicator receipt), those of skill in the art will understand that any or all of these method steps can be performed by other devices in an analyte monitoring system, such as, for example, a local computer system, a wearable computing device, or a medication delivery device. It will also be understood by those of skill in the art that method 1000 of FIG. 10A can combined with any of the other methods described herein, including but not limited to method 700 of FIG. 7, relating to expired and or failed sensor transmissions.

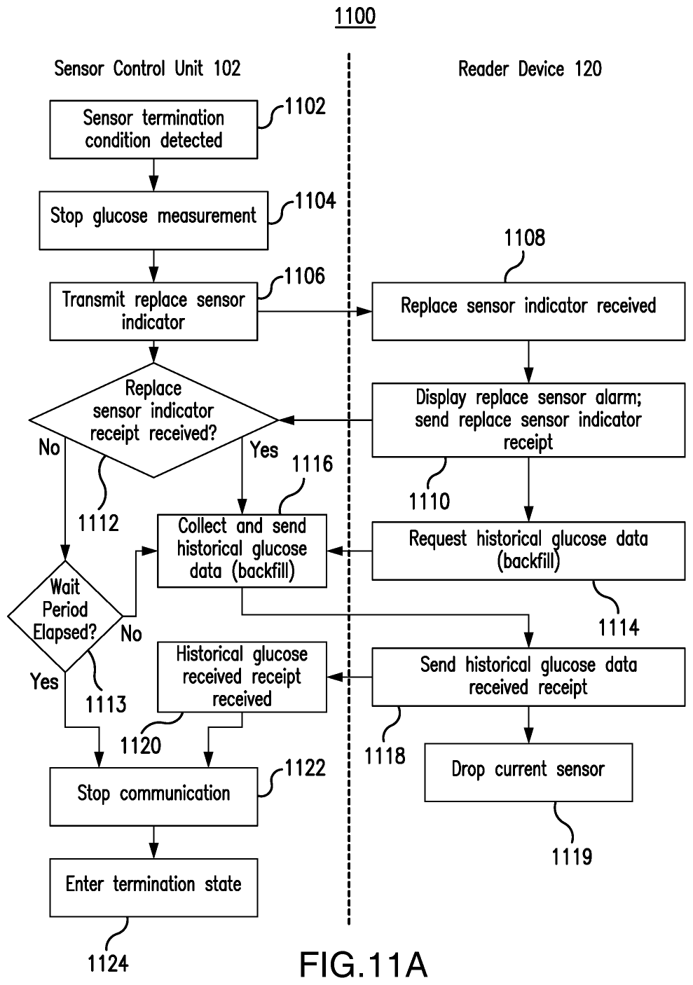

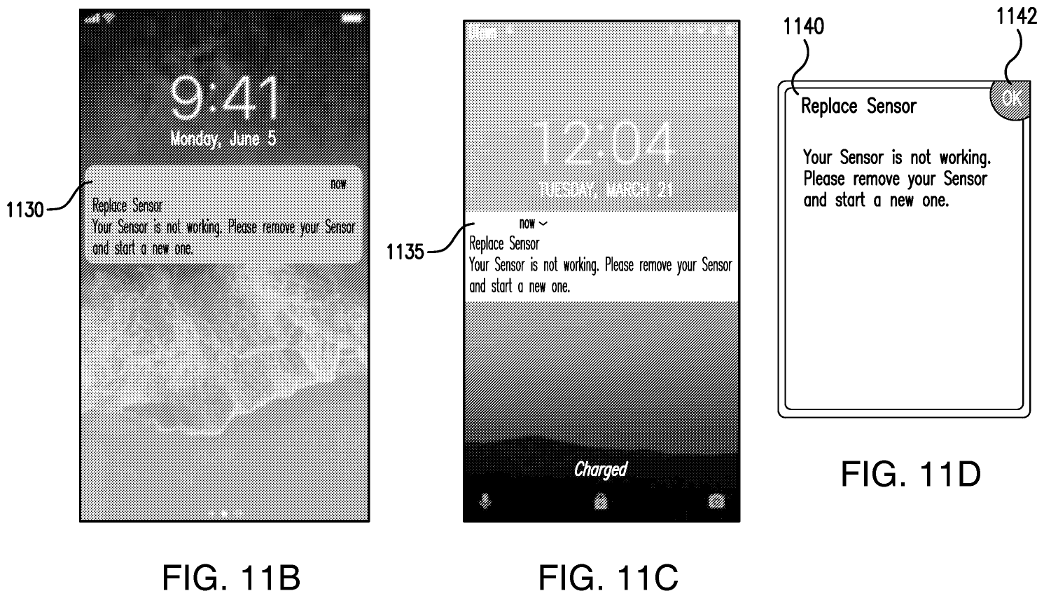

FIG. 11A is a flow diagram depicting an example embodiment of a method 1100 for generating a sensor termination system alarm (also referred to as a “replace sensor” system alarm). At Step 1102, a sensor termination condition is detected by sensor control device 102. As described earlier, a sensor termination condition can include, but is not limited to, one or more of the following: a FIFO overflow condition detected, a sensor signal below a predetermined insertion failure threshold, moisture ingress detected, an electrode voltage exceeding a predetermined diagnostic voltage threshold, an early signal attenuation (ESA) condition, or a late signal attenuation (LSA) condition, to name a few.

At Step 1104, in response to the detection of a sensor termination condition, sensor control device 102 stops taking glucose measurements. At Step 1106, sensor control device 102 generates a replace sensor indicator and transmits it via wireless communication circuitry to reader device 120. Subsequently, at Step 1112, sensor control device 102 will continue to transmit the replace sensor indicator while determining whether a replace sensor indicator receipt has been received from reader device 102. In accordance with the disclosed subject matter, sensor control device 102 can continue to transmit the replace sensor indicator until either: (1) a predetermined waiting period has elapsed (Step 1113), or (2) a receipt of the replace sensor indicator is received (Step 1112) and sensor control device 102 has successfully transmitted backfill data (Steps 1116, 1120) to reader device 120.

Referring still to FIG. 11A, if a wireless communication link is established between sensor control device 102 and reader device 120, then reader device 120 will receive the replace sensor indicator at Step 1108. In response to receiving the replace sensor indicator, reader device 120 will display a replace sensor system alarm at Step 1110. FIGS. 11B to 11D are example embodiments of replace sensor system alarm interfaces, as displayed on reader device 120. In some embodiments, for example, the replace sensor system alarm can be a notification box, banner, or pop-up window that is output to a display of a smart phone, such as interfaces 1130 and 1135 of FIGS. 11B and 11C. In some embodiments, the check sensor alarm can be output to a display on a reader device 120, such as a glucose meter or a receiver device, such as interface 1140 of FIG. 11D. According to the embodiments, to acknowledge receipt of the indicator, reader device 120 can also transmit a replace sensor indicator receipt back to sensor control device 102. In some embodiments, for example, the replace sensor indicator receipt can be automatically generated and sent upon successful display of the replace sensor system alarm 1130, 1135, or 1140. In other embodiments, the replace sensor indicator is generated and/or transmitted in response to a predetermined user input (e.g., dismissing the check sensor system alarm, pressing a confirmation ‘OK’ button 1142, etc.).

At Step 1114, after displaying the replace sensor system alarm and transmitting the replace sensor indicator receipt, reader device 120 can then request historical glucose data from sensor control device 102. At Step 1116, sensor control device 102 can collect and send to reader device 120 the requested historical glucose data. In accordance with the disclosed subject matter, the step of requesting, collecting, and communicating historical glucose data can comprise a data backfilling routine, such as the methods described with respect to FIGS. 6A and 6B.

Referring again to FIG. 11A, in response to receiving the requested historical glucose data, reader device 120 can send a historical glucose data received receipt to sensor control device 102 at Step 1118. Subsequently, at Step 1119, reader device 120 drops sensor control device 102. In accordance with the disclosed subject matter, for example, Step 1119 can comprise one or more of: terminating an existing wireless communication link with sensor control device 102; unpairing from sensor control device 102; revoking an authorization or digital certificate associated with sensor control device 102; creating or modifying a record stored on reader device 120 to indicate that sensor control device 102 has been terminated; or transmitting an update to trusted computer system 180 to indicate that sensor control device 102 has been terminated.

At Step 1120, sensor control device 102 receives the historical glucose data received receipt. Subsequently, at Step 1122, sensor control device 102 stops the transmission of the replace sensor indicator and, at Step 1124, sensor control device 102 can enter into a termination state in which sensor control device 102 does not take glucose measurements and the wireless communication circuitry is either de-activated or in a dormant mode. In accordance with the disclosed subject matter, when in a termination state, sensor control device 102 cannot be re-activated by reader device 120.

Although method 1100 of FIG. 11A is described with respect to glucose measurements, those of skill in the art will appreciate that sensor control device 102 can be configured to measure other analytes (e.g., lactate, ketone, etc.) as well. In addition, although method 1100 of FIG. 11A describes certain method steps performed by reader device 120 (e.g., receiving replace sensor indicator, displaying a replace sensor system alarm, and sending a replace sensor indicator receipt), those of skill in the art will understand that any or all of these method steps can be performed by other devices in an analyte monitoring system, such as, for example, a local computer system, a wearable computing device, or a medication delivery device. It will also be understood by those of skill in the art that method 1100 of FIG. 11A can combined with any of the other methods described herein, including but not limited to method 700 of FIG. 7, relating to expired and or failed sensor transmissions.

### Example Embodiments of Data Integration With Electronic Health Medical Records

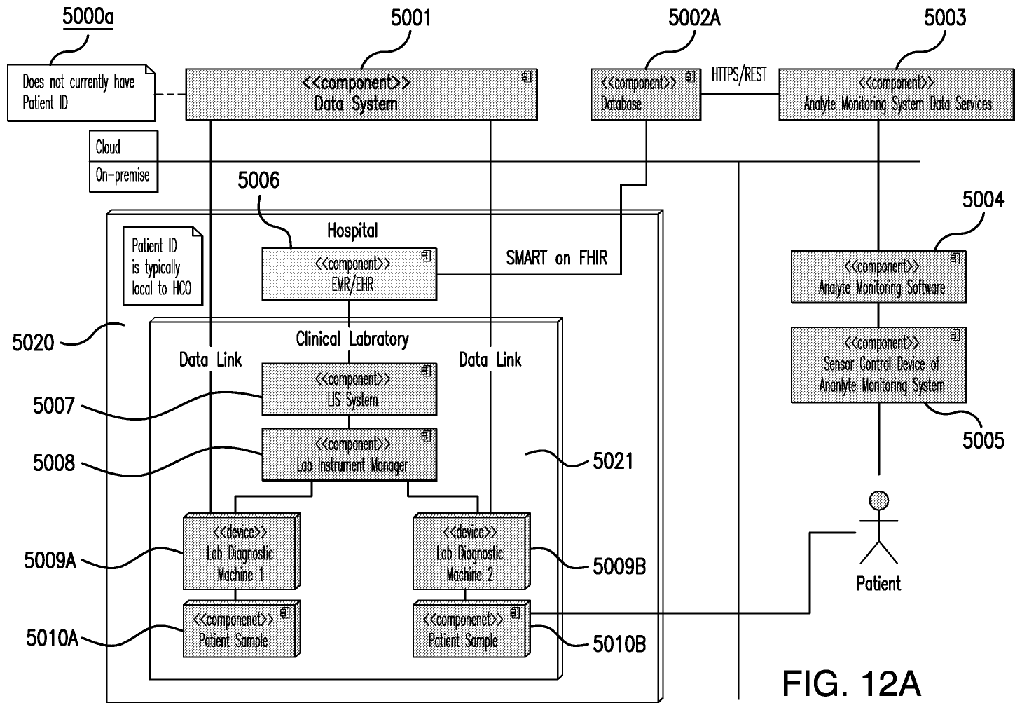

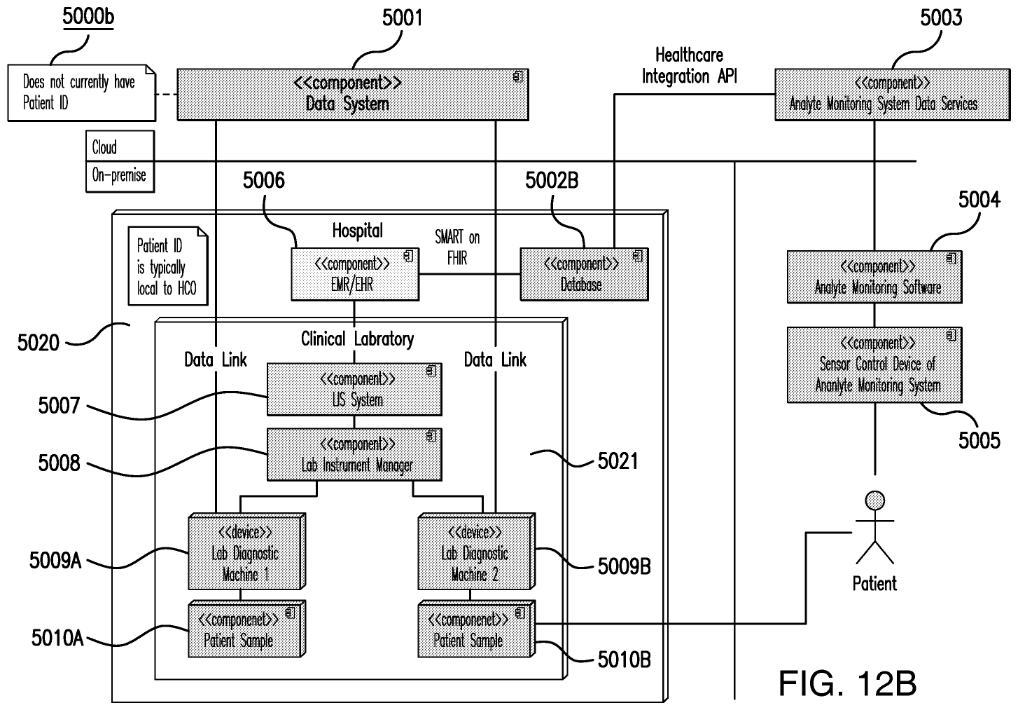

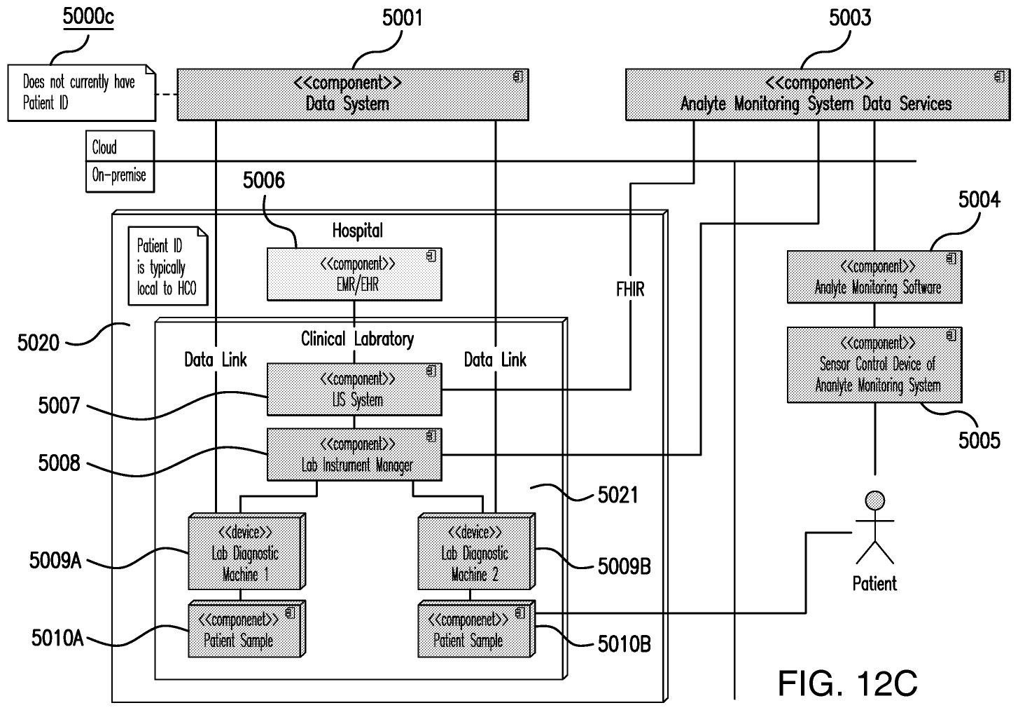

Described herein are example embodiments of systems and methods for bi-directional communication of patient data. According to one aspect of the embodiments, as shown in FIG. 12A, system 5000a for bi-directional communication of patient data can include a database (e.g., hospital or health care organization (HCO)) 5020, another database 5002A, and data services 5003 (e.g., in some embodiments, analyte monitoring system data services).

Analyte monitoring system data services 5003 can be a trusted computer system 180, as described above, and can include a cloud-based server or network including software to provide services including, for example and without limitation, authentication and user profile management, secured data transmission and storage, and analyte data report generation. Analyte monitoring software 5004 can be a user interface application (e.g., software) such as those described above, and can be a reader device 120. According to embodiments, data services 5003 can store analyte measurements generated and transmitted by a plurality of reader devices and sensor control devices in communication with data service 5003. According to embodiments, data service 5003 maintains a record of stored analyte measurements by associating them with appropriate user ID. For example, not limitation, user ID can include email address, date of birth, first name, last name, address, geographic location of the patient, social security number, phone number, etc. or any combination thereof.

According to embodiments, as can be seen in FIG. 12A, hospital 5020 can include an electronic medical/health records component 5006 in communication with clinical laboratory 5021. Clinical laboratory 5021 can include a laboratory information system or LIS system 5007, lab instrument manager 5008, and one or more lab diagnostic machines 5009A, B (two shown). According to embodiments, one or more lab diagnostic machines 5009A, B can communicate with data system 5001 using a data link, which can include a wired or wireless technique. One or more lab diagnostic machines 5009A, B are configured to receive patient sample 5010A, B, respectively, and perform laboratory analysis on the received samples. In operation, electronic medical/health records component 5006 generates and sends an order to the LIS system 5007 for performance of laboratory analysis for a particular patient. The LIS system 5007 sends the order to lab instrument manager 5008, which sends the order to the appropriate lab diagnostic machines 5009A, B. Once a patient sample is received by lab diagnostic machines 5009A, B, laboratory analysis is performed and the results from the laboratory analysis are transmitted to the lab instrument manager 5008, which transmits the results to LIS system 5007, which in turn transmits the results to the electronic medical/health records 5006.

According to embodiments, patient sample 5010A, B can include, without limitation, any other biological fluid or sample known to a person of ordinary skill in the art, such as blood, urine, etc. According to embodiments, laboratory analysis can include, without limitation, analyzing how well organs such as kidneys, liver, thyroid, heart, etc. are working. For example, not limitation, results of the laboratory analysis can include a patient's HbA1c, cholesterol, lipid panel, CBC, etc.

According to embodiments, the electronics medical/health records (EMR) 5006 generates a sample ID corresponding to a personal identification of a patient (or patient ID). Thereafter, the sample ID is transmitted in conjunction with the generated order to the LIS system 5007. As such, the patient ID remains specific to the EMR 5006 and sample ID alone is associated with the order within clinical laboratory 5021 environment. Once laboratory analysis is complete and results from the laboratory analysis are transmitted to the electronics medical/health records 5006, electronics medical/health records 5006 pairs the sample ID associated with the results to the appropriate patient ID. Accordingly, a record of the results from the laboratory analysis can be associated with a patient ID. For example, not limitation, patient ID can include email address, date of birth, first name, last name, address, geographic location of the patient, social security number, phone number, etc. or any combination thereof. Patient ID represents a unique identification metric for each patient at the HCO. In some embodiments, patient ID is specific to each hospital. Therefore, the same patient may not have the same patient ID across different HCOs. In other words, the same patient can have different patient IDs at different HCOs.

As can be seen in FIG. 12A, database 5002A can be a database maintained on a remote cloud server in communication with data services 5003 and electronics medical/health records 5006 and can include one or more processors (not shown). Database 5002A can receive a record of results from the laboratory analysis along with the associated patient ID from EMR 5006 and a record of analyte measurements along with the associated user ID from data services 5003. Thereafter, one or more processors can pair the results from the laboratory analysis with the analyte measurements based upon a shared data item contained in the records. For example, not limitation, if a patient ID matches with a user ID, then laboratory results associated with the patient ID can be paired with the analyte measurements associated with the user ID. For example, not limitation, shared data item can include email address, date of birth, first name, last name, address, geographic location of the patient, social security number, phone number, etc. or any combination thereof. One or more processors can further receive a request to read, write, edit, or delete a resource data in the first or second database, wherein the request is formatted according to a Fast Healthcare Interoperability Resources (FHIR) standard and FHIR extensions embodying a healthcare provider directory (HPD) standard, or H7.

According to embodiments, one or more processors of database 5002A can perform a calculation based on the laboratory results and the analyte measurements. For example, not limitation, where the laboratory results include HbA1c and the analyte measurements include glucose measurements, the one or more processors can perform a calculation of a glucose derived A1c or a kinetic model for determining A1c. Additional details of calculation of a glucose-derived A1c or a kinetic model for determining A1c are set forth in U.S. Patent Publication No. 2018/0235524 to Dunn et al., International Publication No. WO 2020/086934 to Xu, U.S. Provisional Patent Application No. 62/939,970, U.S. Provisional Patent Application No. 63/015,044, U.S. Provisional Patent Application No. 63/081,599, U.S. Provisional Patent Application No. 62/939,956, U.S. Provisional Patent Application No. 63/015,044, and U.S. Provisional Patent Application No. 63/081,599, each of which is incorporated by reference in its entirety herein.

According to embodiments, database 5002A can communicate with electronics medical/health records 5006 according to a Fast Healthcare Interoperability Resources (FHIR) standard, such as those disclosed in U.S. Patent Publication No. 20017/0270532, which is incorporated herein in its entirety. In some embodiments, database 5002A can communicate with electronics medical/health records 5006 using SMART on FHIR. According to embodiments, database 5002A can communicate with data services 5003 according to a Hypertext Transfer Protocol Secure (HTTPS) or REpresentational State Transfer (REST) protocol.

According to embodiments, as can be seen in FIG. 12B, database 5002B is similar to database 5002A in that database 5002B is in communication with data services 5003 and electronics medical/health records 5006 and can include one or more processors (not shown). Database 5002B is similar to database 5002A in all aspects except that database 5002B can be located at hospital 5020, rather than on a remote cloud server, and that database 5002B can communication with data services 5003 using a healthcare integration API (as shown), rather than HTTPS or REST. Accordingly, because database 5002B resides on premises at hospital 5020, database 5002B can communicate with EMRs 5006 that may not have capabilities to communicate with databases located on a remote cloud server, for example and without limitation due to network firewalls, network policy configurations, and/or lack of VPN capabilities). Additionally, all records received by one or more processors now resides on premises on hospital 5020, therefore mitigating privacy and data integration concerns because records associated with patient ID will not be sent outside hospital 5020.

According to embodiments, as can be seen in FIG. 12B, data services 5003 can directly communicate with EMR 5006 or lab instrument manager 5008 thereby eliminating the need for database 5002a and b (as shown in FIGS. 12A-B). As such, data services 5003 can include one or more processors that perform the same functions as one or more processors on database 5002a as discussed above. Additionally, data services 5003 can communicate with electronics medical/health records 5006 according to a Fast Healthcare Interoperability Resources (FHIR) standard, such as those disclosed in U.S. Patent Publication No. 20017/0270532, which is incorporated herein in its entirety. In some embodiments, database 5002A can communication with electronics medical/health records 5006 using SMART on FHIR. According to embodiments, data service 5003 can communicate with EMR 5006 such that records from EMR 5006 or lab instrument manager 5008 can be received using blockchain technologies. According to embodiments, details of suitable digital passes and corresponding verification systems and methods are set forth in U.S. Pat. No. 10,991,158 to Luthra et al., which is incorporated herein in its entirety.

In accordance with the disclosed subject matter, a system for managing patient health, wellness, and more is provided comprising a sensor that is configured to be positioned at least in part in contact with the interstitial fluid in a body of a user. In one embodiment, the system can manage diabetes and use a glucose sensor 104. The system further includes sensor electronics 160 configured to be coupled to the glucose sensor and process data indicative of a plurality of monitored glucose levels from the glucose sensor. The system further includes a network 190 comprising one or more servers and at least one processor configured to receive the processed data and receive or store the processed data in a database such as 5002A or 5002B, wherein the processed data is associated with the user. The database 5002A and 5002B can be on a server, multiple servers, or on a distributed server network such as network 190 including one or more cloud servers. The system further includes a reader device 120 configured to receive the processed data from the sensor electronics and the server 180 receives the processed data from the reader device. The system further includes one or more processors configured to create a blockchain, record on the blockchain the first record, including the first data and the associated personal identification. The one or more processors are further configured to record on the blockchain the second record, the second record including the second data and the associated personal identification. The one or more processors are further configured to access the recorded first record on the first blockchain, pair on the blockchain the first data and the second data based upon a shared data item contained in the first record and the second record.

In accordance with the disclosed subject matter, a system for bi-directional communication of patient data using the blockchain is provided comprising a first database having a first record including first data associated with a personal identification of a patient and a second database having a second record including second data associated with a user identification of the patient. The system further includes one or more processors configured to create a blockchain, record on the blockchain the first record, including the first data and the associated personal identification. The one or more blockchains can be implemented on a server, multiple servers, or on a distributed server network such as network 190 including one or more cloud servers. The one or more processors are further configured to record on the blockchain the second record, the second record including the second data and the associated personal identification. The one or more processors are further configured to access the recorded first record on the first blockchain, pair on the blockchain the first data and the second data based upon a shared data item contained in the first record and the second record.

In accordance with the disclosed subject matter, a method for bi-directional communication of patient data is provided comprising receiving from a diagnostic system, using one or more processors, a first data, receiving from a user, using the one or more processors, a personal identification associated with the first data, creating, using the one or more processors, a blockchain, recording, using the one or more processors, a first record on the blockchain, the first record including the first data and the personal identification associated with the first data, accessing, using the one or more processors, the recorded first record on the blockchain, receiving, using the one or more processors, a second record including a second data associated with a user identification from the second database, pairing, using the one or more processors, the first data and the second data on a block of the blockchain based upon a shared data item contained in the first record and the second record; and displaying, using one or more processors, a combination of the first data and the second data.

By using the blockchain in this manner, the bi-directional communication of patient data will be improved. On the blockchain, the two data records are paired based on a shared data item contained in the first record and the second record. The shared data item is an email address, name and date of birth, a public cryptographic key, or any other unique identifying information. Further in accordance with the disclosed subject matter, the system can include a first database that is an electronic medical record system, and a first data that is laboratory measured HbA1c. The system can further include a second database that is an analyte monitoring system data service, and second data that is glucose levels measured by an analyte monitoring system. The system can further generate a notification based upon the first data paired with the second data, wherein the notification is displayed as the combination of the first data with the second data. Because the system enables the use of FHIR standards, including FHIR extensions embodying a healthcare provider directory (HPD) standard, or H7, the system allows for programmable hooks that could be linked to one or more data sets. The system could further allow these hooks to be programmed using SMART applications to be plugged into the EMR or EHR 5006 system of the provider, including being provided through the EMR or EHR 5006 system. The system can further perform a calculation based upon the first data paired with the second data, wherein the calculation includes a glucose derived A1c, or a personalized HbA1c.

Further in accordance with the disclosed subject matter, the method may include a first database first database that is an electronic medical record system, and a first data that is laboratory measured HbA1c. The method can further include a second second database that is an analyte monitoring system data service, and second data that is glucose levels measured by an analyte monitoring system. The method can further comprise generating a notification based upon the first data paired with the second data. The method can further perform a calculation based upon the first data paired with the second data, which can include a glucose derived A1c or a personalized HbA1c. In a further embodiment, the notifications can be directed to a user with requests for reported outcome measures, such as to identify any preceding activities or other factors that could be matched with the first data and second data to provide further insight into the health of the monitored patient. The blockchain in another embodiment is enhanced by associating other types of patient recorded information with the analyte monitoring events. For example, if an identified glucose derived A1c is outside the expected range, a notification can be triggered to direct the patient with a response to ask how the patient is doing, thus providing additional context to help physicians improve management issues that require more patient-directed management.

A further description regarding the blockchain technologies is described herein for illustrative purposes. A blockchain based database allows the storing of records using public and private keys, wherein the private key is unique to a user. An advantage of a blockchain database includes immutable characteristics once the transaction record has been updated on the blockchain. The blockchain database includes a distributed transaction ledger storing information that is accessible by databases 5002A or 5002B. Due to the nature of the decentralized ledger, blockchain transactions are immutable. Confirmed transactions of the blockchain use cryptography to ensure that the integrity of the ledger can be verified by any particular node on the network. Blocks on the blockchain may include a block ID and data content.

As discussed above, database 5002A can receive a record of results from the laboratory analysis along with the associated patient ID. As also further discussed above, each Hospital may generate a different patient ID for results for a patient. Additionally, analyte measurements from data services use an associated user ID, but multiple analyte measurement systems may have different user IDs. These user IDs may not be associated with each other. At the blockchain, each user's record would associate each user ID and patient ID with a user. A user's record at the blockchain thus would have a full listing of every associated user ID and patient ID. The blockchain record will be used to link the results for every patient ID coming from EMR 5006 and every user ID coming from data services 5003. This will allow integration of records for disparate hospitals and analyte measurement services or systems. To allow the patient ID to be linked to a particular user, a request can be made to the user at the hospital to seek consent to share patient identifying information with the blockchain database. In a further embodiment, any provider system could make the request to the patient for authorizing or sharing of the patient generated health data. The request made at the hospital is a non-limiting example of how the patient request can be made to share the patient generated health data.

### Example Embodiments of Digital Interfaces for Combined Data From Analyte Monitoring Systems and Electronic Health Records

According to embodiments, a report of the combined data from analyte monitoring systems and laboratory results from EMRs can be received by HCPs, caregivers, and/or analyte monitoring system users. According to embodiments, with SMART-FHIR, based applications, the provider could also access the report or a dashboard with the report directly through the EMR system. According to embodiments, HCPs may receive a different report or “dashboard” from caregivers and/or users. For example, not limitation, HCPs may receive a detailed report showing the combined data. Examples of a detailed report can include graphical representation of analyte measurements over a period of time, overlayed with laboratory measurements (e.g., in certain embodiments, HbA1c), graphical representation with various icons representing different laboratory results. According to embodiments, period of time can be selected by HCPs and can include, without limitation, 1 day, 5 days, 7 days, 14 days, 2 weeks, 1 month, 3 months, or any other period of time that may be clinically relevant. The HCP can use the combined data to provide patients with targets, for example, without limitation, “HbA1c level of 6.4% on your next visit.”

According to embodiments, the user may receive insights or encouragement based on the combined data. For example, not limitation, a user may receive a notification. Notifications can include, without limitation, “Based on your laboratory results and analyte measurements, we predict your HbA1c to be X.” According to embodiments, the notification may additionally state, “If you exercise/change diet/etc., your HbA1c level may lower to Y.”

According to embodiments, the combined data can be used in conjunction with any of the graphical user interfaces described above. According to embodiments, the user can personalize any of the graphical interfaces described above to additionally display data received from EMR 5006.

While the disclosed subject matter is described herein in terms of certain illustrations and examples, those skilled in the art will recognize that various modifications and improvements may be made to the disclosed subject matter without departing from the scope thereof. Moreover, although individual features of one embodiment of the disclosed subject matter may be discussed herein or shown in the drawings of one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for the purposes of illustration and description. It is not intended to be exhaustive or to limit the disclosed subject matter to those embodiments disclosed.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

1. A system for managing diabetes configured to receive data from one or more glucose sensors, the data indicative of glucose levels of a user, the system comprising:
one or more processors configured to:
receive a first record including first data associated with a personal identification of a patient from a first database;
receive a second record including second data associated with a user identification of the patient from a second database;
create a blockchain;
record on the blockchain the first record, the first record including the first data and the associated personal identification;
record on the blockchain the second record, the second record including the second data and the associated user identification;
access the recorded first record on the blockchain; and
pair on a block of the blockchain the first data and the second data based upon a shared data item contained in the first record and the second record.

2. (canceled)

3. The system of claim 1, wherein the first database is an electronic medical record system.

4. The system of claim 1, wherein the first data is laboratory measured HbA1c.

5. The system of claim 1, wherein the second database includes an analyte monitoring system data service.

6. The system of claim 1, wherein the second data includes glucose levels measured by an analyte monitoring system.

7. The system of claim 1, wherein the shared data item includes an email address.

8. The system of claim 1, wherein the shared data item includes a public key.

9. The system of claim 1, further comprising a server comprising the one or more processors.

10. The system of claim 1, further comprising a distributed server network comprising the one or more processors.

11. A system for bi-directional communication of patient data comprising:
one or more processors configured to:
receive a first record including first data associated with a personal identification of a patient from a first database;
receive a second record including second data associated with a user identification of the patient from a second database; and
create a blockchain;
record on the blockchain the first record, the first record including the first data and the associated personal identification;
record on the blockchain the second record, the second record including the second data and the associated personal identification;
access the recorded first record on the first blockchain; and
pair on a block of the blockchain the first data and the second data based upon a shared data item contained in the first record and the second record.

12. The system of claim 11, wherein the first database is an electronic medical record system.

13. The system of claim 11, wherein the first data is laboratory measured HbA1c.

14. The system of claim 11, wherein the second database includes an analyte monitoring system data service.

15. The system of claim 11, wherein the second data includes glucose levels measured by an analyte monitoring system.

16. The system of claim 11, wherein the shared data item includes an email address.

17. The system of claim 11, wherein the shared data item includes a public key.

18. The system of claim 11, wherein the one or more processors is further configured to generate a notification based upon the first data paired with the second data.

19. The system of claim 18, wherein the notification is displayed as the combination of the first data paired with the second data.

20. The system of claim 11, wherein the one or more processors is further configured to perform a calculation based upon the first data paired with the second data.

21. The system of claim 20, wherein the calculation includes calculation of a glucose derived A1c.

22. The system of claim 20, wherein the calculation includes calculation of a personalized HbA1c.

23. A method of bi-directional communication of patient data comprising:
receiving from a diagnostic system, using one or more processors, a first data;
receiving from a user, using the one or more processors, a personal identification associated with the first data;
creating, using the one or more processors, a blockchain;
recording, using the one or more processors, a first record on the blockchain, the first record including the first data and the personal identification associated with the first data;
accessing, using the one or more processors, the recorded first record on the blockchain;
receiving, using the one or more processors, a second record including a second data associated with a user identification from the second database;
pairing, using the one or more processors, the first data and the second data on a block of the blockchain based upon a shared data item contained in the first record and the second record; and
displaying, using one or more processors, a combination of the first data and the second data.

24. The method of claim 23, wherein the second database is an electronic medical record system.

25. The method of claim 23, wherein the first data is laboratory measured HbA1c.

26. The method of claim 23, wherein the second database includes an analyte monitoring system data service.

27. The method of claim 23, wherein the second data includes glucose levels measured by an analyte monitoring system.

28. The method of claim 23, wherein the shared data item includes an email address.

29. The method of claim 23, wherein the shared data item includes a public key.

30. The method of claim 23, further comprising generating, using the one or more processors, a notification based upon the first data paired with the second data.

31. The method of claim 23, further comprising performing, using the one or more processors, a calculation based upon the first data paired with the second data.

32. The method of claim 31, wherein the calculation includes calculation of a glucose derived A1c.

33. The method of claim 31, wherein the calculation includes calculation of a personalized HbA1c.

34. The system of claim 1, wherein the first database is an electronic medical record system, wherein the first data is laboratory measured HbA1c, wherein the second data includes glucose levels measured by an analyte monitoring system, and wherein the one or more processors are further configured to perform a calculation based upon the first data paired with the second data.

35. The system of claim 34, wherein the one or more processors are further configured to trigger a notification when the calculation is outside of an excepted range.

36. The method of claim 31, further comprising triggering a notification when a result of the calculation is outside an expected range.
